(19) United States
(12) Patent Application Publication
(10) Pub. No.: US 2016/0370374 A1
(43) Pub. Date: Dec. 22, 2016

(54) GLYCOMIC PATTERNS FOR THE DETECTION OF DISEASE

(71) Applicant: Massachusetts Institute of Technology, Cambridge, MA (US)

(72) Inventors: Carlos Bosques, Arlington, MA (US); Ram Sasisekharan, Bedford, MA (US); Sasi Raguram, Hillsborough, NJ (US)

(73) Assignee: Massachusetts Institute of Technology, Cambridge, MA (US)

(21) Appl. No.: 15/078,905

(22) Filed: Mar. 23, 2016

Related U.S. Application Data
(63) Continuation of application No. 11/732,482, filed on Apr. 3, 2007, now abandoned.
(60) Provisional application No. 60/789,026, filed on Apr. 3, 2006.

(51) Int. Cl. G01N 33/574

(52) U.S. Cl. CPC G01N 33/57484; G01N 33/57434; G01N 33/57407; G01N 2440/38

(57) ABSTRACT

This invention relates, in part, to methods and products for the detection of cancer, such as prostate cancer or multiple myeloma. This invention also relates, in part, to methods and products for the detection of prostate disease, such as benign prostatic hyperplasia (BPH). This invention further relates, in part, to methods and products for the detection of specific glycans in one or more samples, such as, for example, methods whereby specific glycans are detected and their amounts analyzed. Such methods can be used to determine relative ratios and/or threshold values for the specific glycans described herein. The relative ratios and/or threshold values can be used in the methods provided.

### RELATED APPLICATIONS

This application is a continuation application of U.S. patent application Ser. No. 11/732,482, filed Apr. 3, 2007, which claims the benefit under 35 U.S.C. §119(e) from U.S. provisional application No. 60/789,026, filed Apr. 3, 2006, each of which is incorporated by reference herein in its entirety.

### GOVERNMENT SUPPORT

Aspects of this invention may have been made using funding from National Institutes of Health grant numbers GM 057073 and U54 GM62116 as well as National Institutes of Health/National Institute of Environmental Health Sciences grant numbers ES002109 and 5-T32-ES0720. Accordingly, the government may have rights in the invention.

### FIELD OF THE INVENTION

This invention relates, in part, to methods and products for the detection of cancer, such as prostate cancer or multiple myeloma. This invention also relates, in part, to methods and products for the detection of prostate disease, such as benign prostatic hyperplasia (BPH). This invention further relates, in part, to methods and products for the detection of specific glycans in one or more samples, such as, for example, methods whereby specific glycans are detected and their amounts analyzed. Such methods can be used to determine relative ratios and/or threshold values for the specific glycans described herein. These relative ratios and/or threshold values can be used in the methods provided.

### BACKGROUND OF THE INVENTION

Detection of diseases, such as cancers, at an early stage is beneficial for efficient treatment. For the last three decades, major progress has been made in the design of new therapies against cancer. However, survival rates have only been significantly increased for early diagnosed patients. Despite advances in diagnostic technologies, many cases of cancer are not diagnosed and treated until the malignant cells have invaded the surrounding tissue or metastasized throughout the body. Although current diagnostic approaches have significantly contributed to the detection of cancer, they still present problems in their predictive value. Therefore, the discovery of new biomarkers and technologies that can help in this important endeavor is of value.

One drawback of standard clinical proteomics is the deficiency in analyzing post-translational modifications¹ despite their large abundance and important roles in diverse biological processes.^{2,3} Protein glycosylation is one of the most common post-translational modifications in humans. In fact, most proteins destined to be secreted are glycosylated,⁴⁻⁸ including important tumor biomarkers, such as the prostate-specific antigen (PSA)⁹ and the ovarian cancer marker CA125.¹⁰ Expressed on the cell surface and in the extracellular matrix, glycans are important participants in microenvironment remodeling during tumorigenesis. For example, N-glycans have been associated with each and every aspect of tumor progression, from growth and proliferation to angiogenesis and metastasis.³ In the same manner that the underexpression, truncation and altered branching patterns of certain glycans facilitate cell growth during development, they can enhance the capacity of tumors to proliferate.³ N-glycans are also involved in the suppression of apoptosis by modulating the activity of insulin-like growth factor-1 receptors.¹¹ In particular, upregulation of sialyltransferases and N-acetylglucosaminyltransferase V (which results in increased sialylation and branching of N-linked glycans, respectively) are hallmarks of different aspects of tumorigenesis.^{12,13} Increased sialylation on the cell surface may, for example, promote cell detachment from primary tumor via charge repulsion.^{3,14} On the other hand, increased branching on N-linked glycans has been implicated in, in some instances, invasion,¹⁵ angiogenesis and metastasis.¹²

### SUMMARY OF THE INVENTION

Provided herein are methods for detecting glycans in one or more samples. Also, provided are methods of diagnosis and methods for assessing progression or regression through the detection of one or more glycans in a sample from a subject.

In one aspect of the invention a method of diagnosis is provided. The method of diagnosis can, in some embodiments, comprise determining the amount of one or more sialylated glycans in a sample and comparing the amount of the one or more sialylated glycans with a threshold value. In some embodiments, at least one of the one or more sialylated glycans is a NeuAc₃Fuc₁Hex₆HexNAc₅ glycan (e.g., with 3026 [M-H]⁻) or a NeuAc₁Hex₉HexNAc₈ glycan (e.g., with 3391 [M-H]⁻). In other embodiments, the amount of two or more sialylated glycans are determined in a sample and relative ratios of the two or more sialylated glycans are calculated. In some of these embodiments, the methods also include a step of comparing the relative ratios with one or more threshold values. In other embodiments, the two or more sialylated glycans include a NeuAc₃Fuc₁Hex₆HexNAc₅ glycan (e.g., with 3026 [M-H]⁻) and/or a NeuAc₁Hex₉HexNAc₈ glycan (e.g., with 3391 [M-H]⁻). In still further embodiments, the total amount of sialylated glycans, without distinction of the individual species of the sialylated glycans, is determined, and the total amount is compared to a threshold value.

In another aspect of the invention a method of diagnosis is provided comprising determining the amount of one or more glycans selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in a sample, and comparing the amount of the one or more glycans with one or more threshold values.

In still another aspect of the invention a method of diagnosis is provided which comprises determining the amount of a first glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in a sample, determining the amount of a second glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample, calculating the relative ratio of the first glycan and the second glycan, and comparing the relative ratio of the first glycan and the second glycan to a first threshold value.

In some embodiments, the methods provided further comprise determining the amount of a third glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample, determining the amount of a fourth glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample, calculating the relative ratio of the third glycan and the fourth glycan, and comparing the relative ratio of the third glycan and the fourth glycan to a second threshold value.

In other embodiments, the first glycan is a NeuAc2Hex5HexNAc5 glycan (e.g., with 2426 [M-H]⁻), the second glycan is a NeuAc3Hex7HexNAc6 glycan (e.g., with 3245 [M-H]⁻), the third glycan is a NeuAc2Hex6HexNAc5 glycan (e.g., with 2588 [M-H]⁻), and the fourth glycan is a NeuAc3Fuc1Hex6HexNAc5 glycan (e.g., with 3026 [M-H]⁻). In still other embodiments, the first threshold value is 0.112 (or the inverse thereof), and the second threshold value is 0.469 (or the inverse thereof). In yet other embodiments, the first threshold value is 8.9 (or the inverse thereof), and the second threshold value is 2.1 (or the inverse thereof). In some embodiments, the sensitivity of the method is 79%, and/or the specificity of the method is 68%.

In still other embodiments, the first glycan is a NeuAc2Hex5HexNAc4 glycan, the second glycan is a NeuAc2Hex6HexNAc5 glycan, the third glycan is a NeuAc1Fuc1Hex5HexNAc4 glycan, and the fourth glycan is a NeuAc2Hex7HexNAc6 glycan. In further embodiments, the first threshold value is 2.3 (or the inverse thereof), and the second threshold value is 2.3 (or the inverse thereof). In some embodiments, the sensitivity of the method is 79%, and/or the specificity of the method is 70%.

In some embodiments, the methods provided further comprise determining the amount of a fifth glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample, determining the amount of a sixth glycan selected from the group consisting of a NeuAc1 Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample, calculating the relative ratio of the fifth glycan and the sixth glycan, and comparing the relative ratio of the fifth glycan and the sixth glycan to a third threshold value.

In some embodiments, the fifth glycan is a NeuAc3Fuc1Hex6HexNAc5 glycan (e.g., with 3026 [M-H]⁻), and the sixth glycan is a NeuAc1Hex9HexNAc8 glycan (e.g., with 3391 [M-H]⁻). In yet other embodiments, the first threshold value is 0.112 (or inverse thereof), the second threshold value is 0.469 (or inverse thereof), and the third threshold value is 8.035 (or inverse thereof). In still other embodiments, the first threshold value is 8.9 (or inverse thereof), the second threshold value is 2.1 (or inverse thereof), and the third threshold value is 0.1 (or inverse thereof). In some embodiments, the sensitivity of the method is 76%, and/or the specificity of the method is 71%.

In further embodiments, the fifth glycan is a NeuAc2Hex5HexNAc4 glycan (e.g., with 2223 [M-H]⁻), and the sixth glycan is a NeuAc1Hex9HexNAc8 glycan (e.g., with 3391 [M-H]⁻). In some embodiments, the first threshold value is 0.112 (or inverse thereof), the second threshold value is 0.469 (or inverse thereof), and the third threshold value is 7.905 (or inverse thereof).

In yet other embodiments the first glycan is a NeuAc2Hex5HexNAc5 glycan (e.g., with 2426 [M-H]⁻), the second glycan is a NeuAc3Hex7HexNAc6 glycan (e.g., with 3245 [M-H]⁻), the third glycan is a NeuAc3Hex6HexNAc5 glycan (e.g., with 2879 [M-H]⁻), and the fourth glycan is a NeuAc4Hex7HexNAc6 glycan (e.g., with 3536 [M-H]⁻).

In still other embodiments of the methods provided, the methods further comprise determining the amount of a fifth glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample, determining the amount of a sixth glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample, calculating the relative ratio of the fifth glycan and the sixth glycan, and comparing the relative ratio of the fifth glycan and the sixth glycan to a third threshold value.

In yet other embodiments, the fifth glycan is a NeuAc4Fuc1Hex7HexNAc6 glycan (e.g., with 3682 [M-H]⁻), and the sixth glycan is a NeuAc4Hex8HexNAc7 glycan (e.g., with 3902 [M-H]⁻). In some embodiments, the first threshold value is 0.123 (or inverse thereof), the second threshold value is 3.006 (or inverse thereof), and the third threshold value is 4.250 (or inverse thereof).

In another aspect, a method of diagnosing prostate cancer is provided comprising determining the amount of glycans D, A, C, B and E in a sample. In one embodiment, the method further comprises calculating the relative ratio of glycans D and A, the relative ratio of glycans C and B and the relative ratio of glycans E and C. In another embodiment, when the absolute value of the relative ratio of glycans D and A is greater than or equal to 8.9 (D:A) (or less than the inverse of 8.9 (A:D)), the absolute value of the relative ratio of glycans C and B is greater than or equal to 2.1 (C:B) (or less than the inverse of 2.1 (B:C)) and the absolute value of the relative ratio of glycans E and C is greater than or equal to 0.1 (E:C) (or less than the inverse of 0.1 (C:E)), the result is indicative of prostate cancer.

In yet another aspect, a method of diagnosing multiple myeloma is provided comprising determining the amount of glycans F, B, G and H in a sample. In one embodiment, the method further comprises calculating the relative ratio of glycans F and B and the relative ratio of glycans G and H. In another embodiment, when the absolute value of the relative ratio of glycans F and B is less than or equal to 2.3 (F:B) (or greater than the inverse of 2.3 (B:F)) and the absolute value of the relative ratio of glycans G and H is less than or equal to 2.3 (G:H) (or greater than the inverse of 2.3 (H:G)), the result is indicative of multiple myeloma.

In a further aspect of the invention a method of diagnosis is provided comprising determining the relative ratio of tetra-antennary glycans to bi-antennary glycans in a sample, and comparing the relative ratio to a threshold value. In some embodiments, the threshold value is at least 0.6 (or inverse thereof). In other embodiments, the threshold value is 0.6 (or inverse thereof). In still other embodiments, the threshold value is 0.8 (or inverse thereof).

In some embodiments, the methods provided further comprise arriving at a diagnosis. In other embodiments, the diagnosis is a final diagnosis.

In still other embodiments, the methods provided further comprise performing an additional test (e.g., diagnostic test) on the subject. In other embodiments, the additional test is performed on a sample from the subject. In some embodiments, the additional test comprises obtaining another sample from the subject. In other embodiments, the additional test is performed on the same sample as the previous method. In still other embodiments, after an additional test is performed, the method can further comprise arriving at a diagnosis. In some embodiments, the diagnosis is a final diagnosis.

In some embodiments, the additional test comprises determining the amount of one or more additional glycans. In other embodiments, the additional test further comprises comparing the amount of the one or more additional glyans to one or more threshold values. In still other embodiments, the additional test comprises determining the amount of two or more additional glycans, calculating at least one relative ratio of the two or more glycans and comparing the at least one relative ratio with a threshold value. In some embodiments, at least one of the glycans is a sialylated glycan. In other embodiments, the at least one sialylated glycan is a NeuAc₃Fuc₁Hex₆HexNAc₅ glycan (e.g., with 3026 [M-H]⁻) and/or a NeuAc₁Hex₉HexNAc₈ glycan (e.g., with 3391 [M-H]⁻).

In other embodiments, the additional test comprises determining the total amount of sialylated glycans, without distinction of the individual species of sialylated glycans. The total amount is then compared to a threshold value in further embodiments.

In still other embodiments, the additional test, comprises determining the relative ratio of tetra-antennary glycans to bi-antennary glycans, and comparing the relative ratio to a threshold value. In some embodiments, the threshold value is at least 0.6 (or inverse thereof). In other embodiments, the threshold value is 0.6 (or inverse thereof). In further embodiments, the threshold value is 0.8 (or inverse thereof).

In yet other embodiments, the additional test, comprises determining the amount of a prostate cancer-specific marker in the sample, and comparing the amount of the prostate cancer-specific marker to a threshold value. In some embodiments, the prostate cancer-specific marker is prostate-specific antigen (PSA) or PSMA.

In yet further embodiments, the additional test comprises determining the amount of a multiple myeloma-specific marker in the sample, and comparing the amount of the multiple myeloma-specific marker to a threshold value. In some embodiments, the multiple myeloma-specific marker is CD56, CD117 or CD28.

In still other embodiments, the additional test is a digital rectal exam (DRE) or a tissue biopsy. In other embodiments, the additional test is a blood test, urine test, bone marrow test or X-ray.

The methods provided herein, in some embodiments, are performed on a sample obtained from a subject. In some embodiments, the subject is suspected of having cancer. In other embodiments, the subject is suspected of having prostate cancer. In yet other embodiments, the subject is suspected of having multiple myeloma. In still other embodiments, the subject is suspected of having prostate disease. In some embodiments, the prostate disease is BPH.

In a further aspect of the invention a method for analyzing one or more samples is provided. The method can, in some embodiments, comprise determining the amount of one or more sialylated glycans in the one or more samples. In other embodiments, the methods also include determining one or more threshold values from the amounts determined. In some embodiments, at least one of the one or more sialylated glycans is a NeuAc₃Fuc₁Hex₆HexNAc₅ glycan (e.g., with 3026 [M-H]⁻) or a NeuAc₁Hex₉HexNAc₈ glycan (e.g., with 3391 [M-H]⁻). In other embodiments, the amount of two or more sialylated glycans are determined in the one or more samples and relative ratios of the two or more sialylated glycans are calculated. In some of these embodiments, the methods also include a step of determining one or more threshold values from the relative ratios. In other embodiments, the two or more sialylated glycans include a NeuAc₃Fuc₁Hex₆HexNAc₅ glycan (e.g., with 3026 [M-H]⁻) and/or a NeuAc₁Hex₉HexNAc₈ glycan (e.g., with 3391 [M-H]⁻). In still further embodiments, the total amount of sialylated glycans, without distinction of the individual species of sialylated glycans, in the one or more samples is determined. In yet further embodiments, a threshold value for the total amount of sialylated glycans is determined.

In another aspect of the invention a method for determining the amount of one or more glycans selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in one or more samples is provided. In further embodiments, one or more threshold values from the amounts are also determined.

In yet another aspect of the invention a method is provided comprising determining the amount of two or more glycans selected from the group consisting of a NeuAc1 Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in one or more samples. The method, in some embodiments, further includes calculating relative ratios of the glycan amounts in the samples. In yet further embodiments, one or more threshold values from the relative ratios are also determined.

In some embodiments of the methods of detection (and diagnosis, assessing progression, assessing regression, etc.) provided herein, the two or more glycans include a NeuAc2Hex5HexNAc5 glycan (e.g., with 2426 [M-H]⁻) and a NeuAc3Hex7HexNAc6 glycan (e.g., with 3245 [M-H]⁻). In other embodiments, the two or more glycans include a NeuAc2Hex6HexNAc5 glycan (e.g., with 2588 [M-H]⁻) and a NeuAc3Fuc1Hex6HexNAc5 glycan (e.g., with 3026 [M-H]⁻). In still other embodiments, the two or more glycans include a NeuAc3Fuc1Hex6HexNAc5 glycan (e.g., with 3026 [M-H]⁻) and a NeuAc1Hex9HexNAc8 glycan (e.g., with 3391 [M-H]⁻). In yet other embodiments, the two or more glycans include a NeuAc2Hex5HexNAc4 glycan (e.g., with 2223 [M-H]⁻) and a NeuAc1 Hex9HexNAc8 glycan (e.g., with 3391 [M-H]⁻). In still other embodiments, the two or more glycans include a NeuAc3Hex6HexNAc5 glycan (e.g., with 2879 [M-H]⁻) and a NeuAc4Hex7HexNAc6 glycan (e.g., with 3536 [M-H]⁻). In further embodiments, the two or more glycans include a NeuAc4Fuc1Hex7HexNAc6 glycan (e.g., with 3682 [M-H]⁻) and a NeuAc4Hex8HexNAc7 glycan (e.g., with 3902 [M-H]⁻).

In other embodiments, the two or more glycans include a NeuAc2Hex5HexNAc5 glycan (e.g., with 2426 [M-H]⁻), a NeuAc3Hex7HexNAc6 glycan (e.g., with 3245 [M-H]⁻), a NeuAc2Hex6HexNAc5 glycan (e.g., with 2588 [M-H]⁻) and a NeuAc3Fuc1Hex6HexNAc5 glycan (e.g., with 3026 [M-H]⁻). In still other embodiments, the two or more glycans include a NeuAc2Hex5HexNAc5 glycan (e.g., with 2426 [M-H]⁻), a NeuAc3Hex7HexNAc6 glycan (e.g., with 3245 [M-H]⁻), a NeuAc2Hex6HexNAc5 glycan (e.g., with 2588 [M-H]⁻), a NeuAc3Fuc1Hex6HexNAc5 glycan (e.g., with 3026 [M-H]⁻), a NeuAc3Fuc1Hex6HexNAc5 glycan (e.g., with 3026 [M-H]⁻) and a NeuAc1Hex9HexNAc8 glycan (e.g., with 3391 [M-H]⁻). In still further embodiments, the two or more glycans include a NeuAc2Hex5HexNAc5 glycan (e.g., with 2426 [M-H]⁻), a NeuAc3Hex7HexNAc6 glycan (e.g., with 3245 [M-H]⁻), a NeuAc2Hex6HexNAc5 glycan (e.g., with 2588 [M-H]⁻), a NeuAc3Fuc1Hex6HexNAc5 glycan (e.g., with 3026 [M-H]⁻), a NeuAc2Hex5HexNAc4 glycan (e.g., with 2223 [M-H]⁻) and a NeuAc1Hex9HexNAc8 glycan (e.g., with 3391 [M-H]⁻). In other embodiments, the two or more glycans include a NeuAc2Hex5HexNAc5 glycan (e.g., with 2426 [M-H]⁻), a NeuAc3Hex7HexNAc6 glycan (e.g., with 3245 [M-H]⁻), a NeuAc3Hex6HexNAc5 glycan (e.g., with 2879 [M-H]⁻) and a NeuAc4Hex7HexNAc6 glycan (e.g., with 3536 [M-H]⁻). In further embodiments, the two or more glycans include a NeuAc2Hex5HexNAc5 glycan (e.g., with 2426 [M-H]⁻), a NeuAc3Hex7HexNAc6 glycan (e.g., with 3245 [M-H]⁻), a NeuAc3Hex6HexNAc5 glycan (e.g., with 2879 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻). In some embodiments, the two or more glycans include a NeuAc2Hex5HexNAc4 glycan (e.g., with 2223 [M-H]⁻) and a NeuAc2Hex6HexNAc5 glycan (e.g., with 2588 [M-H]⁻). In other embodiments, the two or more glycans include a NeuAc1Fuc1Hex5HexNAc4 glycan (e.g., with 2078 [M-H]⁻) and a NeuAc2Hex7HexNAc6 glycan (e.g., with 2953 [M-H]⁻). In yet other embodiments, the two or more glycans include a NeuAc2Hex5HexNAc4 glycan (e.g., with 2223 [M-H]⁻), a NeuAc2Hex6HexNAc5 glycan (e.g., with 2588 [M-H]⁻), a NeuAc1Fuc1Hex5HexNAc4 glycan (e.g., with 2078 [M-H]⁻) and a NeuAc2Hex7HexNAc6 glycan (e.g., with 2953 [M-H]⁻).

In yet another aspect of the invention a method for analyzing one or more samples is provided which comprises determining the amount of tetra-antennary glycans and bi-antennary glycans in the samples. In some embodiments, the methods further include calculating relative ratios of tetra-antennary glycans to bi-antennary glycans in the samples. In still further embodiments, the methods also include determining one or more threshold values from the relative ratios.

In some embodiments, the one or more samples are from subjects with cancer. In other embodiments, the cancer is prostate cancer. In further embodiments, the cancer is multiple myeloma. In yet other embodiments, the one or more samples also include one or more samples from subjects that do not have cancer. In still other embodiments, the one or more samples also include one or more samples from subjects that do not have cancer or prostate disease.

In other embodiments, the one or more samples are from subjects with prostate disease. In some embodiments, the prostate disease is BPH. In yet other embodiments, the one or more samples also include one or more samples from subjects that do not have prostate disease. In still other embodiments, the one or more samples also include one or more samples from subjects that do not have cancer or prostate disease.

In a further aspect, a method for determining the stage of cancer is provided which comprises determining the amount of a first glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻) a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in a sample, and determining the amount of a second glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample. In some embodiments, the method further comprises calculating the relative ratio of the first glycan and the second glycan. In other embodiments, the method further comprises comparing the relative ratio of the first glycan and the second glycan to a first threshold value.

In further embodiments, the method further comprises determining the amount of a third glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample, and determining the amount of a fourth glycan selected from the group consisting of a NeuAc1Hex5HexNAc4 glycan (e.g., with 1932 [M-H]⁻), a NeuAc2Hex4HexNAc4 glycan (e.g., with 2061 [M-H]⁻), a NeuAc1Fuc1Hex5HexNAc4 glycan (e.g., with 2078 [M-H]⁻), a NeuAc1Hex5HexNAc6 glycan (e.g., with 2177 [M-H]⁻), a NeuAc2Hex5HexNAc4 glycan (e.g., with 2223 [M-H]⁻), a NeuAc1Fuc1Hex4HexNAc6 glycan (e.g., with 2323 [M-H]⁻), a NeuAc2Fuc1Hex5HexNAc4 glycan (e.g., with 2370 [M-H]⁻), a NeuAc2Hex5HexNAc5 glycan (e.g., with 2426 [M-H]⁻), a NeuAc2Fuc1Hex5HexNAc5 glycan (e.g., with 2572 [M-H]⁻), a NeuAc2Hex6HexNAc5 glycan (e.g., with 2588 [M-H]⁻), a NeuAc2Fuc1Hex6HexNAc5 glycan (e.g., with 2735 [M-H]⁻), a NeuAc1Fuc2Hex5HexNAc7 glycan (e.g., with 2834 [M-H]⁻), a NeuAc3Hex6HexNAc5 glycan (e.g., with 2879 [M-H]⁻), a NeuAc2Hex7HexNAc6 glycan (e.g., with 2953 [M-H]⁻), a NeuAc1Fuc3Hex5HexNAc7 glycan (e.g., with 2980 [M-H]⁻), a NeuAc3Fuc1Hex6HexNAc5 glycan (e.g., with 3026 [M-H]⁻), a NeuAc3Fuc1Hex6HexNAc6 glycan (e.g., with 3228 [M-H]⁻), a NeuAc3Hex7HexNAc6 glycan (e.g., with 3245 [M-H]⁻), a NeuAc1Hex9HexNAc8 glycan (e.g., with 3391 [M-H]⁻), a NeuAc4Hex7HexNAc6 glycan (e.g., with 3536 [M-H]⁻), a NeuAc4Fuc1Hex7HexNAc6 glycan (e.g., with 3682 [M-H]⁻) and a NeuAc4Hex8HexNAc7 glycan (e.g., with 3902 [M-H]⁻) in the sample. In some embodiments, the method also comprises calculating the relative ratio of the third glycan and the fourth glycan. In further embodiments, the method also comprises comparing the relative ratio of the third glycan and the fourth glycan to a second threshold value.

In one embodiment, the first glycan is a NeuAc2Hex5HexNAc5 glycan (e.g., with 2426 [M-H]⁻), the second glycan is a NeuAc3Hex7HexNAc6 glycan (e.g., with 3245 [M-H]⁻), the third glycan is a NeuAc3Fuc1Hex6HexNAc5 glycan (e.g., with 3026 [M-H]⁻), and the fourth glycan is a NeuAc2Hex6HexNAc5 glycan (e.g., with 2588 [M-H]⁻). In another embodiment, the first threshold value is 9.8 (or the inverse thereof), and the second threshold value is 3.5 (or the inverse thereof).

In another aspect, a method of determining the stage of prostate cancer is provided comprising determining the amount of glycans D, A, C and B in a sample. In one embodiment, the method further comprises calculating the relative ratio of glycans D and A and the relative ratio of glycans C and B. In another embodiment, when the value of the relative ratio of glycans D and A is greater than or equal to 9.8 (D:A) (or less than the inverse of 9.8 (A:D)) and the value of the relative ratio of glycans C and B is greater than 3.5 (C:B) (or less than the inverse of 3.5 (B:C)), the result is indicative of Stage III prostate cancer. In one embodiment, the values are absolute values.

In another embodiment, the subject has or is thought to have prostate cancer.

In a further aspect, a method for determining the stage of cancer is provided comprising determining the relative ratio of tetra-antennary glycans to bi-antennary glycans in the sample, and comparing the relative ratio to a threshold value to determine the stage of cancer in the subject. In one embodiment, the threshold value is at least 0.8 (or the inverse thereof). In another embodiment, the subject has or is thought to have prostate cancer.

In some embodiments, the samples are serum, saliva, urine, seminal fluid or tissue samples.

In other embodiments, applicable to any of the methods provided herein, determining the amount a glycan refers to determining the total amount of the glycan in the sample and not just the amount of the glycan from a particular glycoprotein. In still other embodiments, the total amount of the glycan in the sample is determined after high abundance proteins (e.g., immunoglobulins, albumin and/or transferrin) are removed.

In other embodiments, the NeuAc1Hex5HexNAc4 glycan (e.g., with 1932 [M-H]⁻) is NeuAc1Hex5HexNAc4 (e.g., with 1932 [M-H]⁻), the NeuAc2Hex4HexNAc4 glycan (e.g., with 2061 [M-H]⁻) is NeuAc2Hex4HexNAc4 (e.g., with 2061 [M-H]⁻), the NeuAc1Fuc1Hex5HexNAc4 glycan (e.g., with 2078 [M-H]⁻) is NeuAc1Fuc1Hex5HexNAc4 (e.g., with 2078 [M-H]⁻), the NeuAc1Hex5HexNAc6 glycan (e.g., with 2177 [M-H]⁻) is NeuAc1Hex5HexNAc6 (e.g., with 2177 [M-H]⁻), the NeuAc2Hex5HexNAc4 glycan (e.g., with 2223 [M-H]⁻) is NeuAc2Hex5HexNAc4 (e.g., with 2223 [M-H]⁻), the NeuAc1Fuc1Hex4HexNAc6 glycan (e.g., with 2323 [M-H]⁻) is NeuAc1Fuc1Hex4HexNAc6 (e.g., with 2323 [M-H]⁻), the NeuAc2Fuc1Hex5HexNAc4 glycan (e.g., with 2370 [M-H]⁻) is NeuAc2Fuc1Hex5HexNAc4 (e.g., with 2370 [M-H]⁻), the NeuAc2Hex5HexNAc5 glycan (e.g., with 2426 [M-H]⁻) is NeuAc2Hex5HexNAc5 (e.g., with 2426 [M-H]⁻), the NeuAc2Fuc1Hex5HexNAc5 glycan (e.g., with 2572 [M-H]⁻) is NeuAc2Fuc1Hex5HexNAc5 (e.g., with 2572 [M-H]⁻), the NeuAc2Hex6HexNAc5 glycan (e.g., with 2588 [M-H]⁻) is NeuAc2Hex6HexNAc5 (e.g., with 2588 [M-H]⁻), the NeuAc2Fuc1Hex6HexNAc5 glycan (e.g., with 2735 [M-H]⁻) is NeuAc2Fuc1Hex6HexNAc5 (e.g., with 2735 [M-H]⁻), the NeuAc1Fuc2Hex5HexNAc7 glycan (e.g., with 2834 [M-H]⁻) is NeuAc1Fuc2Hex5HexNAc7 (e.g., with 2834 [M-H]⁻), the NeuAc3Hex6HexNAc5 glycan (e.g., with 2879 [M-H]⁻) is NeuAc3Hex6HexNAc5 (e.g., with 2879 [M-H]⁻), the NeuAc2Hex7HexNAc6 glycan (e.g., with 2953 [M-H]⁻) is NeuAc2Hex7HexNAc6 (e.g., with 2953 [M-H]⁻), the NeuAc1Fuc3Hex5HexNAc7 glycan (e.g., with 2980 [M-H]⁻) is NeuAc1Fuc3Hex5HexNAc7 (e.g., with 2980 [M-H]⁻), the NeuAc3Fuc1Hex6HexNAc5 glycan (e.g., with 3026 [M-H]⁻) is NeuAc3Fuc1Hex6HexNAc5 (e.g., with 3026 [M-H]⁻), the NeuAc3Fuc1Hex6HexNAc6 glycan (e.g., with 3228 [M-H]⁻) is NeuAc3Fuc1Hex6HexNAc6 (e.g., with 3228 [M-H]⁻), the NeuAc3Hex7HexNAc6 glycan (e.g., with 3245 [M-H]⁻) is NeuAc3Hex7HexNAc6 (e.g., with 3245 [M-H]⁻), the NeuAc1Hex9HexNAc8 glycan (e.g., with 3391 [M-H]⁻) is NeuAc1Hex9HexNAc8 (e.g., with 3391 [M-H]⁻), the NeuAc4Hex7HexNAc6 glycan (e.g., with 3536 [M-H]⁻) is NeuAc4Hex7HexNAc6 (e.g., with 3536 [M-H]⁻), the NeuAc4Fuc1Hex7HexNAc6 glycan (e.g., with 3682 [M-H]⁻) is NeuAc4Fuc1Hex7HexNAc6 (e.g., with 3682 [M-H]⁻) and/or the NeuAc4Hex8HexNAc7 glycan (e.g., with 3902 [M-H]⁻) is NeuAc4Hex8HexNAc7 (e.g., with 3902 [M-H]⁻).

In another aspect of the invention compositions of the glycans described herein are also provided.

In still another aspect of the invention kits comprising reagents (e.g., antibodies, lectins, etc.) for the detection of the glycans described are also provided.

In still a further aspect, forms are provided wherein the values for the amounts or relative ratios of the glycans provided herein are listed. In one embodiment, the form provides values for glycans A, B, C, D, E, F, G and/or H. In another embodiment, the form provides values for glycans D, A, C, B, E and/or C. In yet another embodiment, the form provides values for glycans D, A, C and/or B. In still a further embodiment, the form provides values for glycans F, B, G and/or H. In still another embodiment, the form provides values for the relative ratios of glycans D and A, C and B and/or E and C. In yet another embodiment, the form provides values for the relative ratios of glycans D and A and/or C and B. In still a further embodiment, the form provides values for the relative ratios of glycans F and B and/or G and H. The values can be absolute values in some embodiments. In other embodiments, the form is in written or electronic form.

Each of the limitations of the invention can encompass various embodiments of the invention. It, therefore, is anticipated that each of the limitations of the invention involving any one element or combinations of elements can be included in each aspect of the invention. These and other aspects of the invention will be described in further detail in connection with the detailed description of the invention.

### BRIEF DESCRIPTION OF THE FIGURES

FIG. 1 represents an example of a serum glycomic pattern analysis. The glycans from all glycoproteins in the serum were cleaved and purified. The next step involved analysis of the total mixture of glycans by MALDI-TOF-MS. The complex glycoprofile obtained from the mass spectrometry data was fed into a bioinformatics platform that rapidly identifies patterns associated with a disease or state.

FIG. 2 illustrates the improved sensitivity for the analysis of underivatized acidic glycans with different matrix formulations. The results of a MALDI-TOF-MS analysis of a mixture of 1 pmol neutral and acidic glycan standards using a DHB/spermine matrix analyzed in the negative mode are provided in FIG. 2A. FIG. 2B provides results from a MALDI-TOF-MS analysis of a mixture of 25 fmol neutral and acidic glycan standards using an ATT/Nafion® formulation.

FIG. 3 illustrates improvements in a mass spectra analysis for underivatized sialylated glycans with certain matrix formulations. The results from a MALDI-TOF-MS analysis of a mixture of 10 pmol neutral and acidic glycan standards using DHB/spermine matrix are shown in FIGS. 3A and 3B. Results from a MALDI-TOF-MS analysis of a mixture of 0.1 pmol neutral and acidic glycan standards using an ATT/Nafion® formulation are provided in FIGS. 3C and 3D. A reduction of undesirable peak splitting resulting from multiple ion complexes, reduction of sialic acid cleavage and an elimination of neutral glycan signals in the negative mode are observed.

FIG. 4 provides a schematic representation for the matrix of matrices used to optimize MALDI-TOF-MS analysis of underivatized sialylated glycans.

FIG. 5 illustrates the quantification of acidic glycans with a certain matrix formulation using MALDI-TOF-MS. Correlation between signal intensity, glycan amount and molecular weight is shown. An ATT/Nafion® formulation was used for this analysis. Each glycan was quantified in the presence of 8 other neutral and acidic glycans. An R² value of 0.95 was obtained for the quantification of the acidic glycans.

FIG. 6 illustrates the reproducibility of 27 control samples. The m/z values of 13 samples were recorded for each of the samples. The spectra for each sample in the y-axis is shown with normalized intensity values in the z-axis.

FIG. 7 provides a schematic representation of the bioinformatics approach used for the discovery of disease-associated glycomic patterns.

FIG. 8 illustrates the specificity and sensitivity of a separation of samples of non-cancer patients from cancer patients. ROC curves for the |D/A|≧8.9 and |C/B|≧2.1 rule of the glyco test (solid circles) and total PSA levels (open circles) are shown.

FIG. 9 demonstrates the differences in the glycomic pattern associated with prostate cancer. The MALDI-TOF-MS data for each group of patients illustrate the differences found by the bioinformatics platform. The glycan structures and the observed [M-H]⁻ are shown for each species.

FIG. 10 provides the partial structural analysis of glycans associated with the glycomic PCa patterns. A MALDI-TOF-MS spectra of glycans before treatment with glycosidases in the negative mode, after treatment with non-specific Arthrobacter ureafaciens sialidase A operated in the positive mode, after treatment with bovine kidney fucosidase operated in the positive mode and after treatment with jack-bean β-galactosidase operated in the positive mode are provided in FIGS. 10A, 10B, 10C and 10D, respectively. Bovine kidney fucosidase releases α-1,6 core-linked fucoses more efficiently than other fucoses, such as α-1,3-linked fucoses.

FIG. 11 provides results from a partial structural analysis of glycans associated with glycomic PCa patterns using orthogonal fucosidases. A MALDI-TOF-MS spectra of glycans before treatment with glycosidases operated in the negative mode, after treatment with non-specific Arthrobacter ureafaciens sialidase A operated in the positive mode, after treatment with bovine kidney fucosidase operated in the positive mode and after treatment with almond meal fucosidase operated in the positive mode are provided in FIGS. 11A, 11B, 11C and 11D, respectively. While bovine kidney fucosidase releases α-1,6 core-linked fucoses more efficiently than other fucoses, almond meal fucosidase is specific for α-1,3,4-linked fucoses.

FIG. 12 provides results from a partial structural analysis of glycans associated with glycomic PCa patterns using orthogonal sialidases. A MALDI-MS spectra of glycans before treatment with glycosidases operated in the negative mode, after treatment with non-specific Arthrobacter ureafaciens sialidase operated in the positive mode, after treatment with non-specific Arthrobacter ureafaciens sialidase in the negative mode and after treatment with Streptococcus pneumoniae sialidase operated in the negative mode are provided in FIGS. 12A, 12B, 12C and 12D, respectively. Streptococcus pneumoniae sialidase is specific for α-2,3-linked sialic acids.

FIG. 13 shows differences in the glycomic pattern associated with multiple myeloma. The MALDI-TOF-MS data for each group of patients illustrates the differences found by the bioinformatics platform. The glycan structures and the observed [M-H]⁻ are shown for each species.

### DETAILED DESCRIPTION OF THE INVENTION

Despite the availability of diagnostic tests, improved disease detection, such as cancer detection, would still be beneficial. For example, even with the digital rectal exam (DRE) and the prostate-specific antigen (PSA) test, prostate cancer cases have tripled during the last decade. The PSA test has become a widely used non-invasive measurement for prostate cancer. However, the lack of specificity of this test limits its use for the early diagnosis of prostate cancer. New approaches are needed to improve the detection of prostate cancer, and other cancers and diseases, at an early stage. Described herein are specific glycans and patterns that can serve in the detection of disease, such as cancer (e.g., prostate cancer, multiple myeloma) and prostate disease (e.g., BPH). A method for diagnosing prostate cancer, for example, that has better predictive values than the well-established total PSA test is provided.

Glycans have the potential to be sensitive biomarkers due to their involvement in aspects of tumor progression, for example. Effort has been put into the identification of glycan markers associated with cancer. For example, studies have focused on the characterization of glycans from glycoproteins expressed in cancer cell lines as a mode to identify cancer-associated alterations.^{16,17} This approach, however, is of limited clinical value since the alterations of the glycan structures on a glycoprotein expressed on cells do not reflect the same modifications in human-derived samples, such as serum. For example, it has been recently shown that glycans isolated from PSA expressed in human prostate cancer cell lines (LNCaP cells) are different from the PSA glycans derived from the serum or seminal fluid of a prostate cancer patient.¹⁸ Other approaches for glycan analysis focus on the examination of carbohydrates from a specific glycoprotein as the diagnostic fingerprint. However, correlating the progression of a disease with the exact glycosylation state of a specific glycoprotein has been limited by the pleiotropic effects of glycan remodeling on many systems.³

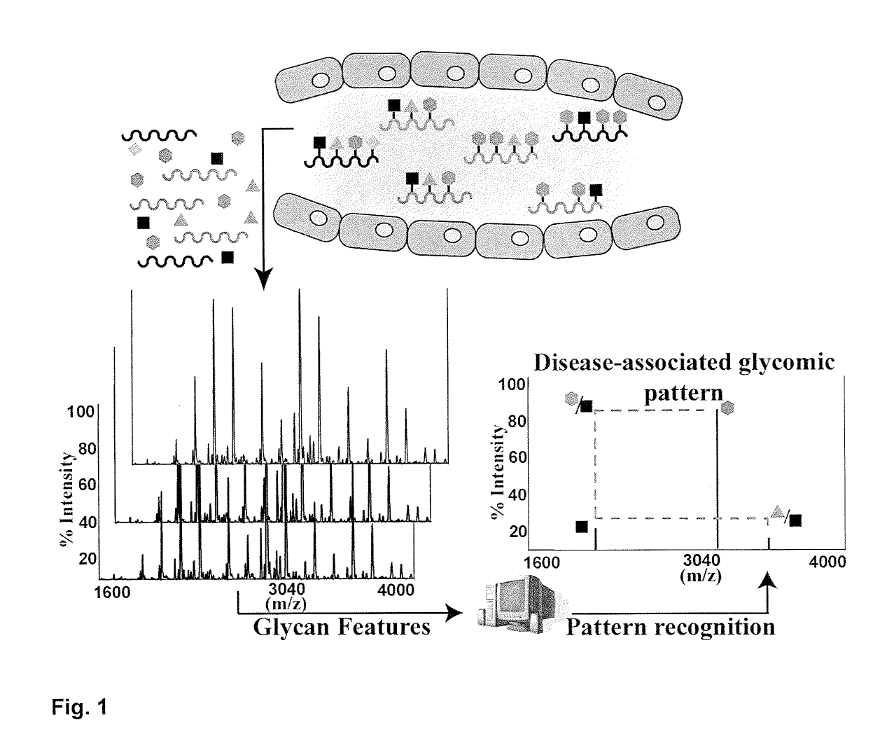

An approach that focuses on using global glycomic patterns from body fluids as a diagnostic fingerprint is described herein and has been provided in U.S. application Ser. Nos. 11/107,982 and 11/244,826. Because of the involvement of glycans in the stages of tumorigenesis, monitoring alterations to the global glycomic patterns in serum could be a more reliable alternative to capture the complex molecular remodeling taking place in the tumor microenvironment. To pinpoint these complex global alterations, while at the same time alleviating the limitations faced by other glycoanalysis technologies, a technique was developed that can rapidly analyze a large number of human serum samples and identify specific glycomic patterns associated with cancer. One example of the above-mentioned approach combines high sensitivity and fast analysis provided by MALDI-TOF-MS with a bioinformatics platform that efficiently extracts meaningful information from large mass spectra data sets. Using this information, the bioinformatics platform then creates rules to rapidly identify glycans as biomarkers (FIG. 1). The method allows for the analysis of a sample population of statistical significance, which is helpful for biomarker discovery, and by focusing on the alterations to global glycomic patterns, this approach can also overcome some of the challenges arising from the pleiotropic effects of glycan remodeling.

Using this method, the sialylated N-glycoprofiles from the serum of 142 patients were analyzed and specific glycomic patterns that distinguish prostate cancer patients from non-cancer donors were identified. Good predictive values were obtained. In fact, better prediction was demonstrated over the well-established total PSA test. The results illustrate the use of global glycomic patterns as diagnostic fingerprints. The results also illustrate that this method, and like approaches, can be used in the discovery of disease-associated glycan biomarkers and opens new possibilities for the use of global glycomic patterns for disease diagnosis.

The study has demonstrated that sialylated glycans (i.e., those that contain a sialic acid, such as, for example, N-acetyl neuraminic acid) can be used in the diagnosis of disease. Therefore, methods for analyzing one or more samples is provided whereby the amount of one or more sialylated glycans is determined. The sialylated glycan can be any glycan that contains a sialic acid. Such glycans include those described throughout the instant specification. For example, the sialylated glycan can be a NeuAc₃Fuc₁Hex₆HexNAc₅ glycan (e.g., with 3026 [M-H]⁻) or a NeuAc₁Hex₉HexNAc₈ glycan (e.g., with 3391 [M-H]⁻). Methods are also provided in which the total amount of sialylated glycans, without distinction of the individual species of the sialylated glycans, is determined. The methods of analyzing sialylated glycans have utility in the diagnosis of disease.

The study conducted has also provided a number of specific glycans, which can be used in the diagnosis of disease, such as cancer and prostate disease. These glycans include any of the glycans presented herein, e.g., in the text immediately following and in Tables 2 and 3, the Examples and figures provided. These glycans include, for example, a NeuAc1Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan. When a specific glycan is recited as shown here, the composition is meant to refer to any glycan with the particular types and numbers of saccharides represented by the composition notation. For example, a “NeuAc3Hex7HexNAc6 glycan” encompasses any glycan that contains 3 N-acetyl neuraminic acids, 7 hexoses and 6 N-acetyl hexosamines. A “NeuAc1Fuc1Hex5HexNAc4 glycan” encompasses any glycan that contains 1 N-acetyl neuraminic acid, 1 fucose, 5 hexoses and 4 N-acetyl hexosamines. These saccharides can be present in any order in the glycan and can be linked to each other with any of a number of types of linkages (e.g., they can be α-1,2-; α-1,6-; α-2,3-; α-2,6-; β-1,2-; β-1,3-; or β-1,4-linked). The term is meant to include these various glycan structures. Further, it will be recognized by one of ordinary skill in the art that the glycans provided may exist in a modified form (e.g., derivatives or enzymatically-modified versions) or a precursor form in the sample or be modified as part of an analytic method (e.g., derivatized, chemically-modified or enzymatically modified) used for its detection. Therefore, the recitation of the specific glycans as provided above include modified and precursor forms, and the methods of detecting one or more of the specifically recited glycans provided herein are meant to include the detection of a modified, a precursor form or any other form from which the amount of the glycan can be inferred.

These glycans above are in some embodiments a NeuAc1Hex5HexNAc4 glycan with 1932 [M-H]⁻, a NeuAc2Hex4HexNAc4 glycan with 2061 [M-H]⁻, a NeuAc1Fuc1Hex5HexNAc4 glycan with 2078 [M-H]⁻ a NeuAc1Hex5HexNAc6 glycan with 2177 [M-H]⁻, a NeuAc2Hex5HexNAc4 glycan with 2223 [M-H]⁻, a NeuAc1Fuc1Hex4HexNAc6 glycan with 2323 [M-H]⁻, a NeuAc2Fuc1Hex5HexNAc4 glycan with 2370 [M-H]⁻, a NeuAc2Hex5HexNAc5 glycan with 2426 [M-H]⁻, a NeuAc2Fuc1Hex5HexNAc5 glycan with 2572 [M-H]⁻, a NeuAc2Hex6HexNAc5 glycan with 2588 [M-H]⁻, a NeuAc2Fuc1Hex6HexNAc5 glycan with 2735 [M-H]⁻, a NeuAc1Fuc2Hex5HexNAc7 glycan with 2834 [M-H]⁻, a NeuAc3Hex6HexNAc5 glycan with 2879 [M-H]⁻, a NeuAc2Hex7HexNAc6 glycan with 2953 [M-H]⁻, a NeuAc1Fuc3Hex5HexNAc7 glycan with 2980 [M-H]⁻, a NeuAc3Fuc1Hex6HexNAc5 glycan with 3026 [M-H]⁻, a NeuAc3Fuc1Hex6HexNAc6 glycan with 3228 [M-H]⁻, a NeuAc3Hex7HexNAc6 glycan with 3245 [M-H]⁻, a NeuAc1Hex9HexNAc8 glycan with 3391 [M-H]⁻, a NeuAc4Hex7HexNAc6 glycan with 3536 [M-H]⁻, a NeuAc4Fuc1Hex7HexNAc6 glycan with 3682 [M-H]⁻ and a NeuAc4Hex8HexNAc7 glycan with 3902 [M-H]⁻. As used herein, a glycan “with 2834 [M-H]⁻” is meant to refer to a glycan that can be determined to have the recited mass with MALDI-TOF-MS in negative mode. It will be understood by one of ordinary skill in the art that the mass recited is approximate and varies according to the reaction conditions and the methods of analysis used. The definition is meant to identify the particular glycan and is not intended to be limited by the specific method of analysis. In some instances glycans are also identified with a specific composition notation preceding the term “glycan”, which is described above. These glycans, therefore, include those with the particular types and numbers of saccharides of the notation provided and can be determined to have the mass recited. Again, the composition notation when preceding “glycan” is meant to refer to any glycan with the saccharides represented in any order and linked by any of a number of types of linkages. In some embodiments, the glycan is one with the saccharides in the order represented. Such glycans are represented without the recitation of “glycan” following the composition notation. For example, in one embodiment the NeuAc1Hex5HexNAc4 glycan is NeuAc1Hex5HexNAc4. In another embodiment, the NeuAc1Hex5HexNAc4 glycan with 1932 [M-H]⁻ is NeuAc1Hex5HexNAc4 with 1932 [M-H]⁻.

The detection of one or more of the glycans provided herein can be used in the diagnosis of a disease. As used herein, “diagnosis” refers to the determination of whether or not a subject has a particular disease, such as cancer or prostate disease. The term is also meant to include instances where the disease in the subject is not finally determined but that further diagnostic testing is warranted. In such embodiments, the method is not by itself determinative of the presence or absence of the disease in the subject but can indicate that further diagnostic testing is needed or would be beneficial. The methods, therefore, can be combined with one or more other diagnostic methods for the final determination of the presence or absence of the disease in the subject. Examples of such other diagnostic methods are described in more detail below. As used herein, a “final determination” or “final diagnosis” refers to ascertaining the presence or absence of the disease in a subject. The final determination or final diagnosis can be the result of any of the methods of the invention, which in some embodiments, can include more than one diagnostic test.

The detection of one or more of the glycans provided herein can also be used to determine the progression or regression of a disease. As used herein, “progression of a disease” refers to the advancement of the disease or worsening of the effects or symptoms of the disease in a subject. As used herein, “regression of a disease” refers to any improvement of the disease or effects or symptoms of the disease in a subject. This term is intended to encompass remission of the disease, any halt in its progression as well as the elimination of the disease (i.e., cure) in the subject. The detection of one or more glycans can also be used, therefore, to determine the stage of a disease in the subject. For example, when the disease is cancer, detection of one or more glycans can be used to determine whether or not the cancer is Stage I, Stage II, Stage III, etc. In one embodiment, the methods provided herein can be used to determine the stage of prostate cancer in a subject. For example, the methods provided can be used to determine whether or not the prostate cancer is Stage III in a subject. As another example, the methods provided can be used to determine whether or not the prostate cancer is Stage I or Stage II in a subject.

The cancer can be any cancer, including melanoma, hepatic adenocarcinoma, prostatic adenocarcinoma or osteosarcoma. Other cancers include biliary tract cancer, bladder cancer, breast cancer; brain cancer including glioblastomas and medulloblastomas; Burkitt's lymphoma, cervical cancer, choriocarcinoma; colon cancer including colorectal carcinomas; endometrial cancer; esophageal cancer, gastric cancer; head and neck cancer, hematological neoplasms including acute lymphocytic and myelogenous leukemia, multiple myeloma, AIDS-associated leukemias and adult T-cell leukemia lymphoma; intraepithelial neoplasms including Bowen's disease; lung cancer including small cell lung cancer and non-small cell lung cancer, lymphomas including Hodgkin's disease and lymphocytic lymphomas; neuroblastomas; oral cancer including squamous cell carcinoma; esophageal cancer; ovarian cancer including those arising from epithelial cells, stromal cells, germ cells and mesenchymal cells; pancreatic cancer, rectal cancer, sarcomas including leiomyosarcoma, rhabdomyosarcoma, liposarcoma, fibrosarcoma, and synovial sarcoma; skin cancer including Kaposi's sarcoma, basocellular cancer, and squamous cell cancer, testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor. In some embodiments, the cancer is prostate cancer. In other embodiments, the cancer is multiple myeloma.

The disease in some embodiments can be any disease of the prostate known in the art. Such diseases include, for example, BPH, prostatitis or prostate cancer. In some embodiments, the prostate disease is BPH.

In some of the methods provided, the step of obtaining a sample from a subject is included. The sample, as used herein, can be any sample from a subject in which one or more of the glycans provided can be detected. The samples can be, for example, a serum, a saliva, an urine, a seminal fluid or a tissue sample.

A “subject”, as used herein, is any human or non-human vertebrate, e.g., dog, cat, horse, cow, pig, monkey, mouse, rat. In some embodiments, the subject is any subject for which the detection of one or more of the glycans provided herein would be beneficial. In one embodiment, the subject is in need of diagnosis.

In some of the methods provided, the step of determining the amount of a glycan is included. “Determining the amount of a glycan” refers to determining the absolute amount of the glycan in the sample or determining the relative amount as compared to, for example, the amount of a standard or another glycan. In one embodiment, the amount of the glycan represents the amount of the glycan from all of the proteins in a sample and not the amount of the glycan from a particular protein. In another embodiment, the amount of the glycan represents the amount of the glycan from the proteins in a sample after high abundance proteins have been removed. This step can be accomplished using the methods provided below in the Examples. In addition, methods for use in detecting or analyzing glycans can also include mass spectrometry, electrophoresis, nuclear magnetic resonance (NMR), chromatographic methods or a combination thereof. Specifically, the mass spectrometric method can be, for example, LC-MS, LC-MS/MS, MALDI-MS, MALDI-TOF, TANDEM-MS or FTMS. The electrophoretic method can be, for example, capillary electrophoresis (CE), and the chromatographic methods can be, for example, HPLC. Furthermore, the methods for use in detecting or analyzing glycans can also include those provided in co-pending U.S. application Ser. Nos. 11/107,982 and 11/244,826. Such methods are incorporated herein by reference.

In another embodiment, the glycans can also be detected and quantified with the use of antibodies. As used herein, the term “antibody” means not only intact antibody molecules but also fragments of antibody molecules retaining specific binding ability. Such fragments are well known in the art and are regularly employed both in vitro and in vivo. The invention, therefore, embraces isolated antibodies or antigen-binding fragments of antibodies having the ability to selectively bind to any of the glycans provided. The present invention also embraces antigen-binding fragments, such as F(ab′)₂, Fab, Fv and Fd fragments. Compositions containing the antibodies or antigen-binding fragments are also provided. Antibodies include polyclonal and monoclonal antibodies, prepared according to conventional methodology.

In still another embodiment, glycans can also be detected and quantified with the use of lectins. Lectins are a well-known family of carbohydrate binding proteins, which are divided into groups according to their carbohydrate specificity (e.g., fucose specific, mannose specific, N-acetylglucosamine specific, galactose/N-acetylglucosamine specific, etc.). Examples of many known lectins are provided in the EY Labs Lectin Catalog (1998), which describes approximately 70 commercially available lectins, and is incorporated herein by reference.

The binding specificity of the antibodies and lectins can be evaluated using, for example, standard Biacore studies and ELISA assays. Such assays can be used to identify the antibodies and lectins that are useful in the methods of the invention. Such assays are also useful for quantifying the amount of a glycan in a sample. Further, the antibodies and lectins can be detectably labeled with e.g., a fluorescent label, radioactive label, chemiluminescent label, etc. Assays for detection of such labels are well known in the art.

Generally, when the amounts of two or more glycans are determined, the relative ratios of the glycans can also be determined. As used herein, a “relative ratio” is the ratio of the absolute or relative amounts of two different glycans. The relative ratio is calculated by dividing the amount of one of the glycans into the amount of the other. The amount of either glycan can be used as the numerator or denominator, and the use of the term is not intended to limit which of the glycans must serve as the numerator or denominator. The relative ratio can given as the absolute value of the result of the division of the two amounts. The two or more glycans can be, for example, any two of the glycans provided herein. For example, the two or more glycans include the following pairs of glycans: a NeuAc3Hex7HexNAc6 glycan (e.g., with 3245 [M-H]⁻) and a NeuAc2Hex5HexNAc5 glycan (e.g., with 2426 [M-H]⁻); a NeuAc3Fuc1Hex6HexNAc5 glycan (e.g., with 3026 [M-H]⁻) and a NeuAc2Hex6HexNAc5 glycan (e.g., with 2588 [M-H]⁻); a NeuAc1Hex9HexNAc8 glycan (e.g., with 3391 [M-H]⁻) and a NeuAc3Fuc1Hex6HexNAc5 glycan (e.g., with 3026 [M-H]⁻); a NeuAc2Hex5HexNAc4 glycan (e.g., with 2223 [M-H]⁻) and a NeuAc1Hex9HexNAc8 glycan (e.g., with 3391 [M-H]⁻); a NeuAc3Hex6HexNAc5 glycan (e.g., with 2879 [M-H]⁻) and a NeuAc4Hex7HexNAc6 glycan (e.g., with 3536 [M-H]⁻); a NeuAc4Fuc1Hex7HexNAc6 glycan (e.g., with 3682 [M-H]⁻) and a NeuAc4Hex8HexNAc7 glycan (e.g., with 3902 [M-H]⁻), etc.

The amounts of one or more glycans or the relative ratios of one or more pairs of glycans can be compared to threshold values. In some embodiments, such a comparison can result in a diagnosis or a determination in regard to the progression or regression of a disease. As used herein, a “threshold value” is a value to which an amount of a glycan or a relative ratio of a pair of glycans in a sample can be compared and is useful in the diagnosis of a disease (e.g., is indicative of the presence or absence of a disease) or is useful in assessing the progression or regression of a disease (e.g., determining the stage of the disease). As an example, the threshold value is the expected amount of a glycan in a sample from a subject with a disease. As another example, the threshold value is the expected relative ratio of a pair of glycans in a sample from a subject with a disease. As a further example, the threshold value is the expected amount of a glycan in a sample or the expected ratio of a pair of glycans in a sample from a subject with a disease at a certain stage (e.g., Stage I, Stage II, Stage III, etc.). In some embodiments, when the amount or relative ratio determined from a sample is greater than or equal to the threshold value presence or absence of a disease, progression or regression of a disease, or the stage of disease is indicated. In other embodiments, when the amount or relative ratio is less than or equal to the threshold value presence or absence of a disease, progression or regression of a disease, or the stage of disease is indicated. Alternatively, the threshold values can be the amounts or relative ratios expected in a sample from a subject that does not have the disease of interest (i.e., a disease free subject or a subject with a different disease but not the one of interest). Comparison with these values can also be used for diagnosis or the assessment of progression or regression of a disease. Furthermore, methods are provided whereby two or more amounts or relative ratios from a sample are compared with two or more threshold values, and it is the comparison with the two or more threshold values in combination that is or is not indicative of a disease or that provides an assessment of the progression or regression of a disease.

Methods are also provided whereby the step of determining one or more threshold values is included. In such methods, for example, the amounts of one or more of the glycans provided herein are determined in one or more samples. The expected amounts or expected relative ratios (e.g., in some instances where the amounts of two or more glycans are determined) and, therefore, the threshold values can then be calculated using the methods provided herein below in the Examples. Other statistical methods for determining the threshold values will be readily apparent to those of ordinary skill in the art. The threshold values can be determined, if necessary, from samples of subjects of the same age, race, gender and/or disease status, etc. In some embodiments, the threshold value is determined from samples from one population of subjects of the same age, race, gender and/or disease status, etc., such as when there are known glycans associated with a disease. In other embodiments, samples from two or more subject populations, wherein the subjects of each of the populations have the same age, race, gender and/or disease status, etc., are analyzed to determine the threshold values. This can be useful, for example, when specific glycans are not yet known to be associated with a disease or further statistical evaluation is required.

It has also been found that the relative ratio of tetra-antennary and bi-antennary glycans can also be used in the diagnosis or determination of progression or regression of disease. Methods are, therefore, provided for determining the relative ratio of tetra-antennary glycans (i.e., glycans with four antennae) and bi-antennary glycans (i.e., glycans with two antennae) in a sample. The methods can, in some embodiments, also include the step of comparing the relative ratio with a threshold value. As used herein, a “threshold value” when used in reference to ratios of tetra-antennary and bi-antennary glycans is intended to refer to an expected value for the ratio that is useful in the diagnosis of a disease or in the assessment of progression or regression of a disease. The ratio determined from a sample can, therefore, be compared to this expected value. Methods are also provided in which the relative ratios are determined in one or more samples as are one or more threshold values from the relative ratios. Such threshold values can be used in the methods provided herein.

As mentioned above, the methods provided herein can further comprise performing another (or additional) test (e.g., diagnostic test) on the subject. The other test can be performed on the same sample from the subject, or the other test can be performed on another sample obtained from the subject. In some embodiments, no samples are involved in the additional test. Examples of this include forms of physical examination.

The additional test can comprise determining the presence or amount of one or more additional glycans in the sample. When the amount of one or more additional glycans are determined, the method can also include the comparison of the one or more amounts with one or more threshold values. When the amounts of two or more additional glycans are determined, the relative ratios of the glycans can be calculated and compared to one or more threshold values. The glycans for which the amounts are determined can be any glycan that may be present in the sample. In some embodiments the glycan is a sialylated glycan. Methods for performing the determination of the presence or amounts of glycans are as provided elsewhere herein.

The additional test, in some embodiments, can comprise determining the total amount of sialylated glycans, without distinction of the individual species of sialylated glycans, in the sample. The total amount can then be compared to a threshold value in some embodiments.

Another example of an additional test is one that comprises determining the relative ratio of tetra-antennary glycans to bi-antennary glycans, and comparing the relative ratio to a threshold value. In some embodiments, the threshold value is at least 0.6. In other embodiments, the threshold value is 0.6. In further embodiments, the threshold value is 0.8. Alternatively, in some embodiments, the threshold value is also determined.

Further examples of additional tests (e.g., diagnostic tests) include determining the presence or amount of a cancer-specific marker in the sample. The term “cancer-specific marker” is a compound differentially associated with a tumor or cancer such that its presence or level of expression can be indicative of the presence or absence of cancer or a tumor in a subject. Examples of cancer-specific markers include HER 2 (p185), CD20, CD33, GD3 ganglioside, GD2 ganglioside, carcinoembryonic antigen (CEA), CD22, milk mucin core protein, TAG-72, Lewis A antigen, ovarian associated antigens such as OV-TL3 and MOv18, high Mr melanoma antigens recognized by antibody 9.2.27, HMFG-2, SM-3, B72.3, PR5C5, PR4D2, and the like. Further examples include MAGE, MART-1/Melan-A, gp100, Dipeptidyl peptidase IV (DPPIV), adenosine deaminase-binding protein (ADAbp), FAP, cyclophilin b, Colorectal associated antigen (CRC)—C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20 and c-erbB-2. In some embodiments, the cancer-specific marker is a prostate cancer-specific marker, such as PSA or PSMA. In other embodiments, the cancer-specific marker is a multiple myeloma-specific marker, such as CD56, CD117 and CD28.

In further embodiments, the amount of a cancer-specific marker can be compared to a threshold value. It will be readily apparent to one of ordinary skill in the art that there are a number of ways to determine the presence or absence or amount of a cancer-specific marker in a sample (e.g., by assaying for the protein or RNA). The amount of protein or RNA may be determined for instance using Northern or Western blot analysis, binding assays, PCR or any other method known to those of skill in the art.

The additional test (e.g., diagnostic) can also be, in some embodiments, a digital rectal exam (DRE) or a tissue biopsy. The additional test (e.g., diagnostic) can also be, in other embodiments, a blood test, urine test, bone marrow test or X-ray. The additional test can also be different variations of the PSA test (e.g., PSA density, PSA velocity, free PSA, complex to total PSA ratio).

The invention also provides kits which can be used to measure the levels of the glycans described herein. In one embodiment, a kit comprises a package containing an antibody or antigen-binding fragment thereof or a lectin that selectively binds to a glycan, and a control for comparing to a measured value of binding. The kit can also include a detectable label. Kits are generally comprised of the following major elements: packaging, an antibody or antigen-binding fragment thereof or a lectin, a control agent and instructions. Packaging may be a box-like structure for holding a vial (or number of vials) containing an antibody or antigen-binding fragment thereof or a lectin, a vial (or number of vials) containing a control agent and instructions. Individuals skilled in the art can readily modify the packaging to suit individual needs. In some embodiments, the control is a threshold value for comparing to the measured value.

Also provided herein are arrays containing the antibodies, antigen-binding fragments thereof or lectins that selectively bind to the glycans described herein. Such arrays can be used in the methods of detection or diagnosis provided. Standard techniques of protein microarray technology can be utilized to analyze the glycans. Protein microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000.

Microarray substrates may include but are not limited to glass, silica, aluminosilicates, borosilicates, metal oxides such as alumina and nickel oxide, various clays, nitrocellulose or nylon. In some embodiments a glass substrate is preferred. In one embodiment, the microarray substrate may be coated with a compound to enhance synthesis of the antibody, antigen-binding fragment or lectin on the substrate. In another embodiment, the antibodies, antigen-binding fragments or lectins are synthesized directly on the substrate in a predetermined grid pattern using methods known in the art. In another embodiment, the substrate may be coated with a compound to enhance binding of the antibody, antigen-binding fragment or lectin to the substrate. In some embodiments, one or more control polypeptides are also attached to the substrate.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Prostate

### Materials and Methods

### Glycan Cleavage and Purification

The proteins and glycoproteins in the serum were denatured by mixing 100 μL of serum with 150 μL of RCM buffer (8 M urea, 3.2 mM EDTA and 360 mM Tris, pH 8.6) and incubated at 37° C. for 30 minutes.³⁴ Proteins were reduced by adding dithiotreitol (DTT) to a final concentration of 0.1 M and incubated for 1 hr at 37° C. Proteins were then carboxymethylated using iodoacetamide (0.5 M final concentration) and incubated at 37° C. in the dark for 1 hour. Denaturing, reducing, and alkylating reagents were then removed, and the buffer was exchanged to 50 mM sodium phosphate buffer pH 7.5 by using 3,000 MWCO spin concentrators at 4° C. N-glycans were selectively released from the glycoproteins by incubation with PNGase F (1,000 U) for 16 hours at 37° C. The glycans were purified using graphitized carbon solid phase extraction (SPE) cartridges (Hypercarb, Thermo Electron Corporation, Waltham, Mass.) using 50% acetonitrile with 0.05% TFA to elute the acidic glycans and dried under vacuum.

### MALDI-TOF-MS Analysis

Purified glycans were dissolved in deionized water, and 1 μL of the sample was mixed with 9 μL of the matrix (10 mg/mL 6-aza-thiothymine in ethanol). The perfluorinated Nafion® resin (1 μL) was spotted on the MALDI probe and allowed to dry under controlled humidity (20 to 25%) before applying 1 μL of the sample/matrix mixture. All MALDI-TOF-MS spectra were acquired with a Voyager-DE STR BioSpectrometry Workstation (PerSeptive Biosystems, Framingham, Mass.) equipped with delayed extraction using the following instrument parameters: accelerating voltage 22 kV, grid voltage 93%, guide wire 0.3% and extraction delay time of 150 ns (unless otherwise noted). All samples were irradiated with a N₂ laser (337 nm) averaging 100 shots/spectrum, and the N-glycans were detected in negative linear (or reflector) mode. A nine-point external calibration was performed using glycan standards for the assignment of ions. Generally, a mass accuracy of <0.1% was obtained using external calibration. As an objective of this study was to determine glycomic patterns from the complex glycoprofile, the data was processed using Data Explorer (Applied Biosystems, Foster City, Calif.) to reduce the noise level prior to the bioinformatics analysis. For this, the advanced baseline correction function of Data Explorer was used followed by the noise removal and Gaussian smoothing functions. Based on the accuracy of the MALDI, all possible compositions were considered that could correspond to ±2 Da from the observed peak. Based on biosynthetic rules and the fact that only acidic glycans should be observed in the negative mode using the optimized conditions, a preliminary composition was assigned. The composition was then further confirmed from the exoglycosidase analysis.

### Samples Used for Prostate Cancer

Samples for the prostate cancer study were acquired through the physician network of Genomics Collaborative Inc. (Cambridge, Mass.), with more than 120,000 patients for their global repository of appropriately consented clinical samples. This was valuable in order to obtain matched controls for prostate cancer and BPH samples. All samples were from American male patients. The controls were matched in race and age to the PCa and BPH patients. The age range of the patients was from 56 to 88 years old. A total of 142 patient samples were used for the PCa study. Of these, 33 were from prostate cancer patients, 38 were from BPH patients and 71 were from healthy patients. From the PCa group, 29 samples were from White/Caucasians patients, 3 were from African-American and 1 was from a Hispanic/Latino patient. For the BPH group, 30 samples were from White/Caucasians patients, 7 were from African-American and 1 was from a Hispanic/Latino patient. Of the healthy patients, 59 were White/Caucasian patients, 10 were from African-American patients and 2 were from Hispanic/Latino patients. Only 3 patients from the PCa group had other types of cancer. Of the 33 PCa samples, 1 was Stage I, 26 were Stage II and 6 were Stage III.

### Total PSA Levels

Total PSA levels were measured using the two-sided sandwich PSA ELISA from Bio Quant (San Diego, Calif.) following the protocol recommended by the manufacturer. Briefly, serum samples (50 μL) were diluted 1:1 with the binding buffer and incubated on the plates for 30 minutes at room temperature. After washing the unbound proteins, the wells were incubated with the anti-PSA horse radish peroxidase (HRP) labeled antibody for 30 minutes at room temperature. After washing the wells, the HRP substrate was added and the absorbance at 450 nm was recorded as a proportional measurement to the PSA concentration. The absorbance was measured using a Molecular Devices Spectra Max 190 plate reader (Sunnyvale, Calif.). Each serum sample was measured in duplicate, and the concentration was determined based on a calibration curve generated using PSA standard solutions provided with the kit.

### Glycosidases Reaction for Glycan Characterization

All glycosidases were purchased from ProZyme (San Leandro, Calif.). Similar conditions were used for the digestion with both sialidases (Arthrobacter ureafaciens sialidase and Streptococcus pneumoniae sialidase). Purified glycans were incubated with 6.5 mU of each enzyme in a final volume of 100 μL of 50 mM sodium phosphate, pH 6.0 at 37° C. and reacted for 48 hours (adding 6.5 mU of enzyme every 24 hours). Digestion of the glycans with almond meal fucosidase was performed at 37° C. in 100 μL of 50 mM sodium acetate, pH 5.0, by adding 3.1 μU of enzyme every 24 hours for a total of 48 hours. Digestion with bovine kidney fucosidase was achieved by treating the glycans with 4.1 mU of enzyme every 24 hours for a total of 48 hours in 100 μL of 100 mM sodium citrate-phosphate buffer containing 50 μg/mL BSA, pH 6.0 at 37° C. Jack bean β-galactosidase digestion was performed in 100 μL of 50 mM sodium citrate-phosphate, pH 3.5, at 37° C. using 15.6 mU of enzyme two times every 24 hours. Glycans were then purified using C-18 and graphitized carbon SPE cartridges.

### Feature Extraction and Classification

All of the computational analysis for feature extraction and classification was performed on a windows platform using C/C++. Automatic peak detection on the mass spectra data was performed via successive elimination of Gaussians starting with the most significant peak. The parameters of the Gaussian were estimated based on the mass spectra signals from glycan standards. Molecular composition and potential structure assignment of the glycans was done based on biosynthetic rules and using the glycan structure database from the Consortium of Functional Glycomics (Cambridge, Mass.). The structural attributes such as branching and fucosylation were derived based on the assignment of the peaks. The rule induction classifier was developed based on the method described by Weiss et al.²⁵ Optimal rules were chosen based on the error rate of the rules on the training set, the performance of the rules on the testing set and the number of variables in the rules.

### Results

### Mass Spectrometry Approach for Glycomic Pattern Analysis

Most studies using MALDI-TOF-MS to analyze carbohydrates mainly focus on characterization and are not significantly affected by the presence of multiple ions or other artifacts often associated with glycan analysis by MALDI-MS. For the analysis of global glycomic pattern alterations described herein, the usual artifacts associated with mass spectrometry analysis of carbohydrates were eliminated and the sensitivity and reproducibility of the method was optimized. Most approaches for improving the sensitivity of carbohydrate MALDI-TOF-MS have focused on the derivatization of the glycans prior to analysis. However, from a diagnostic standpoint, it is preferable to minimize the sample manipulation in order to reduce false variations and artifacts as well as to increase the throughput. To optimize the MALDI-TOF-MS analysis for acidic glycans, a matrix of matrices was generated, targeting the improvement of the following parameters: minimizing multiplicity of peaks for a species due to multiple ion adducts, increasing sensitivity, achieving linear response with respect to glycan amount, minimizing sialic acid cleavage, decreasing signals from neutral glycans in the negative mode and improving spot morphology. The study focused on the analysis of acidic N-linked glycans, and the MALDI-TOF-MS analysis was performed in the negative mode.

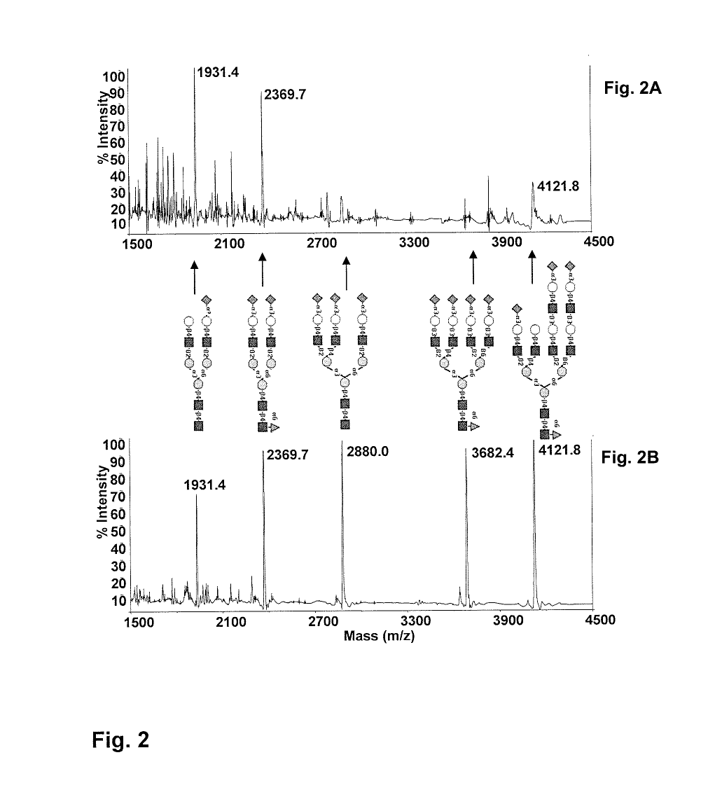

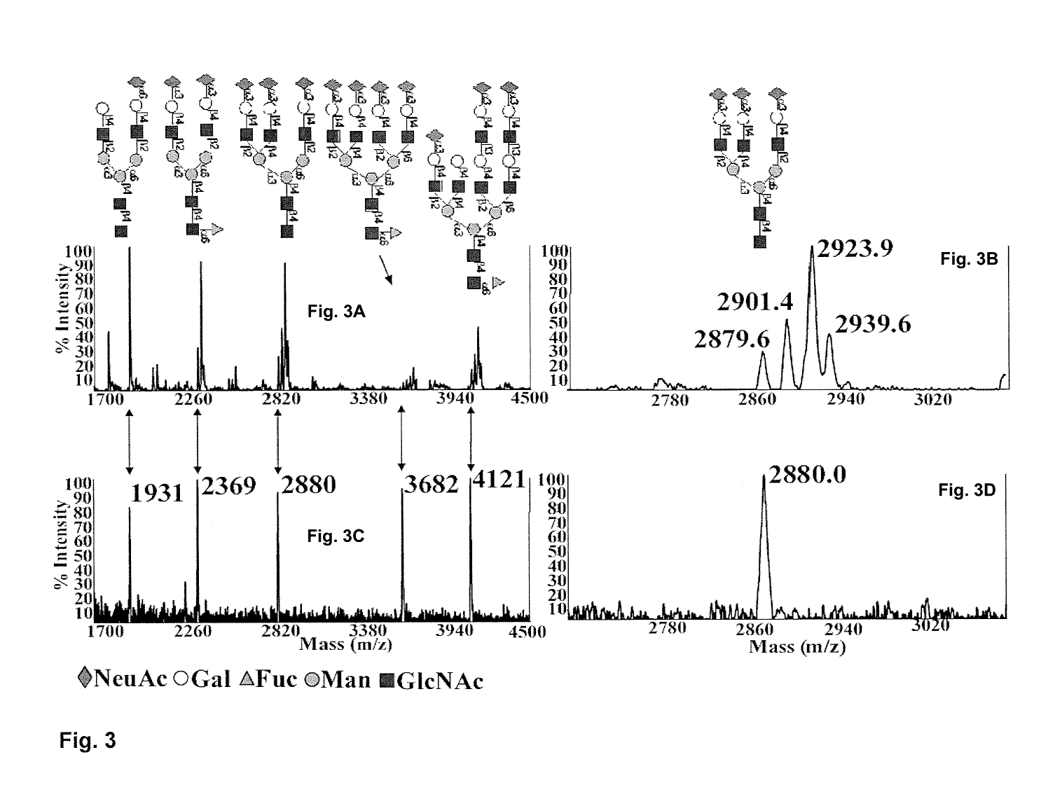

As a starting point, a commonly used matrix for glycans (dihydroxybenzoic acid, DHB) was utilized in combination with spermine (20 mg/ml DHB in acetonitrile and 25 mM spermine in water in a 1:1 ratio). This recipe resulted in detection limits of 10 pmol (FIG. 2) and significant peak splitting with multiple sodium and potassium ions for the acidic glycans (FIG. 3A and FIG. 3B). This matrix also crystallized as long needle-shaped crystals, which complicated the reproducible quantification of glycans present in a sample and eliminated the possibility of automated data acquisition.

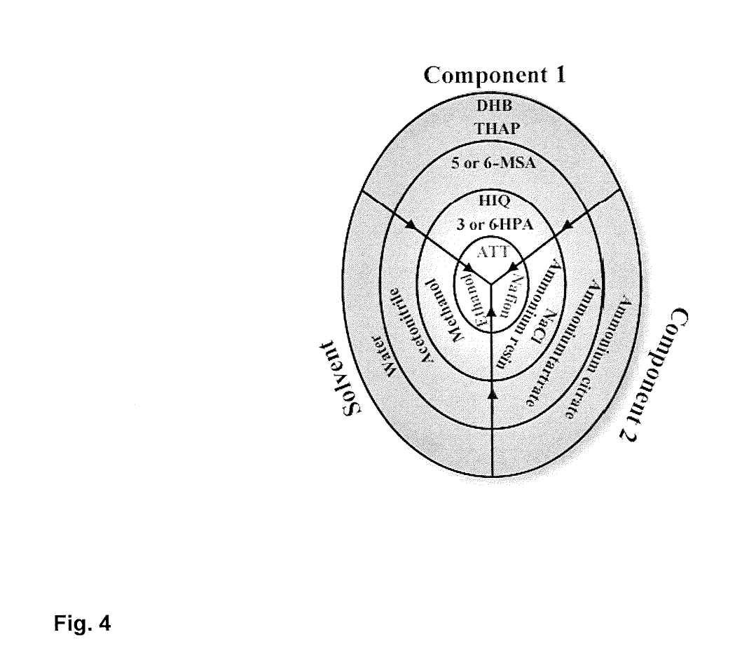

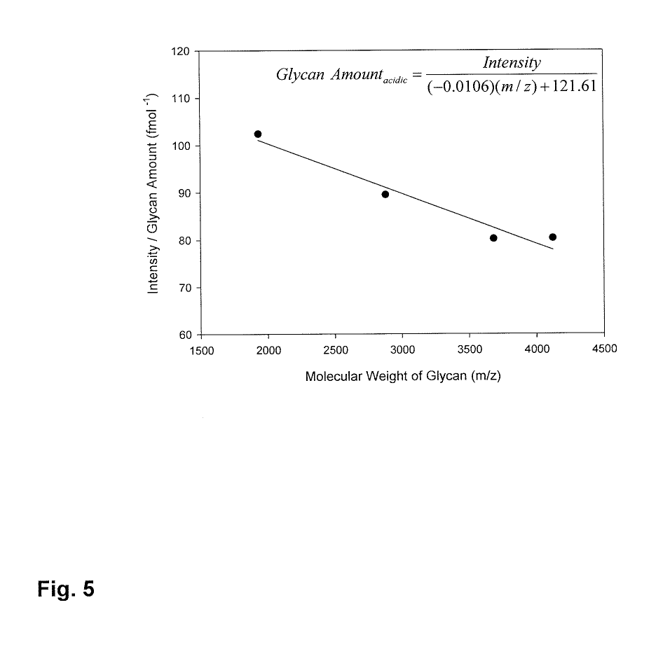

Some of the excipients used for the matrix of matrices optimization included caffeic acid, DHB, spermine, 1-hydroxyisoquinoline (HIQ), 6-aza-2-thiothymine (ATT), 2,4,6-trihydroxyacetophenone (THAP), 6-hydroxypicolinic acid, 3-hydroxypicolinic, 5-methoxysalicylic acid (5-MSA), ammonium citrate, ammonium tartrate, sodium chloride, different ion exchange resins such as ammonium resins and the perfluorinated ion exchange resin Nafion®, etc. These reagents were used in combination with different solvents such as methanol, ethanol, acetonitrile and water. The humidity of the room was also used as another variable for the optimization of conditions. From this study, 6-aza-thiothymine (10 mg/mL in ethanol) spotted on a coating of perfluorinated ion exchange resin (Nafion®) resulted in an optimal recipe (FIG. 4). A controlled room humidity of 20 to 25% also provided optimal results. This matrix recipe achieved complete elimination of peak splitting for the acidic glycans as well as reduction of neutral glycan signals in the negative mode (FIG. 3C and FIG. 3D). This matrix also showed the best detection limits tested for a mixture of underivatized acidic glycans (5 fmol) and showed homogeneous spot morphology and no detectable glycan fragmentation. This recipe also allowed good correlation between signal intensity, glycan amount and molecular weight (FIG. 5). Taken together, these conditions allow a preliminary quantification of glycans present in a mixture, especially at low femtomole quantities, which is an important detection range for possibly clinically relevant species in serum. The optimized conditions were used for subsequent analysis of the acidic N-linked glycans from serum.

### Reproducibility of the Glycomic Profiles

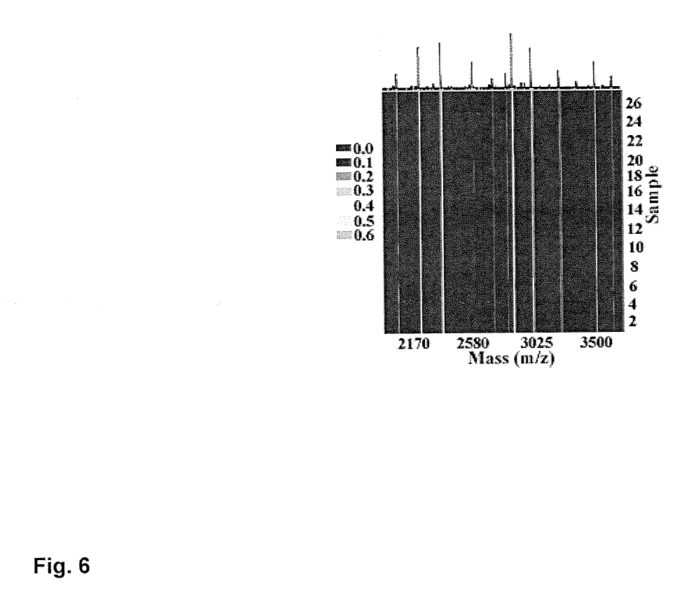

When studying alterations to glycomic profiles by mass spectrometry it is helpful to have good stability of the analytical method in order to decrease the variability associated with other artifacts. The developed method was stable and showed reproducibility for studying the glycoprofile of serum glycans (FIG. 6). To test the precision of the method, 27 control human pooled serum samples (Biomeda, Foster City, Calif.) were processed and analyzed using the optimized conditions. Thirteen peaks across the entire m/z range of the spectra were selected, and the coefficient of variance (CV) was determined for each peak with the normalized intensities (with respect to the total peaks in the spectra). The CV ranged between 6.5 and 19.7% for an average CV of 12.3% for all selected peaks in the 27 serum sample. To study day-to-day variations, 24 control serum samples were analyzed on different days (in groups of 4 samples per day) within a period of 3 months. The coefficient of variance for thirteen selected peaks across the entire m/z range was calculated for the 4 samples in every run. The average CV per run ranged between 5.6 and 16.8% (average CV was 11.3% for all runs). Furthermore, little variation was observed among all 24 independent samples in the different runs. For all independent control samples the average CV was 16.7%. Therefore, both day-to-day and sample-to-sample variations were low. As a comparison, methods used in proteomics pattern diagnostics, where minimum sample manipulation is required during processing and analysis, have shown average coefficient of variance of 10% using 8 selected peaks in 9 spectra.¹⁹ These results show that the method is significantly stable and reliable considering that these measurements reflect the sum of all variations in the total processing and analysis of the sample (i.e., thawing steps, protein denaturation, reduction, carboxymethylation, buffer exchange, deglycosylation, glycan purification, matrix preparations and sample mixing, spotting of samples, mass spectrometry analysis, etc.).

### Bioinformatics Platform for Glycomic Pattern Analysis

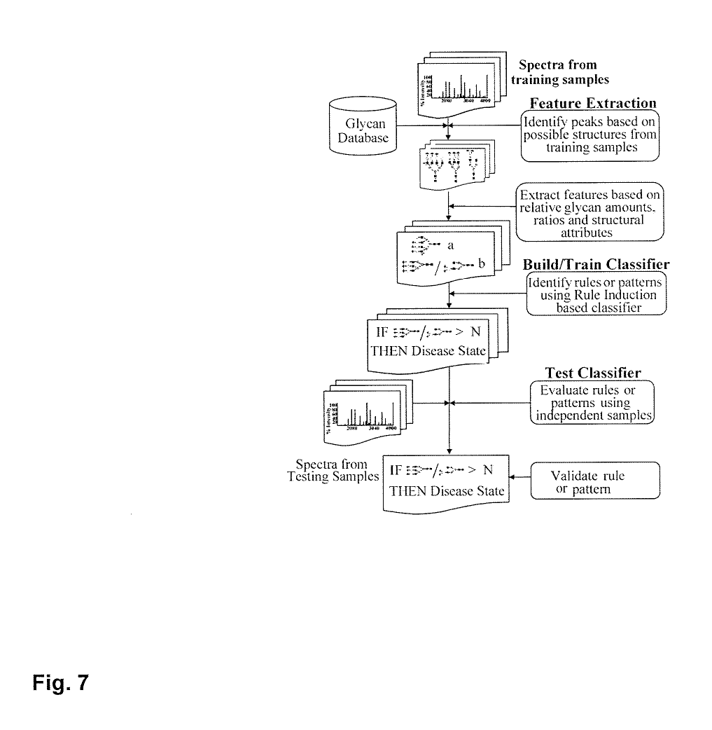

MALDI-TOF-MS analysis can accommodate up to 100 samples in a period of a few hours. However, translating the large and complex information generated from the human serum glycoprofiles into meaningful diagnostic data makes manual analysis difficult. Therefore, bioinformatics methods were developed to identify potential glycan biomarkers in an efficient manner. The design of the bioinformatics platform incorporated some of the inherent properties of glycans, such as their discrete composition and structure. As illustrated in FIG. 7, a three-step approach to identify glycomic patterns that discriminate between samples from diseased and non-diseased patients was implemented by incorporating constraints based on glycan properties and biosynthesis during the process. Features were extracted from the MS-based glycoprofiles. Subsequently, a set of training samples was used to build a classifier²⁴ based on the extracted features. Finally, the classifier was tested using additional samples to verify the predictability of the classifier. During the feature extraction step, peaks were automatically identified in each of the individual mass spectra. The identification process used information from theoretically possible glycan composition based on biosynthetic rules and from the glycan database of the Consortium for Functional Glycomics (Cambridge, Mass.) (functionalglycomics.org/static/consortium). This information was used to guide the peak identification process to ensure that the peaks identified are actual glycans. Three groups of features were generated for each of the mass spectrometry-based glycoprofiles. The first group of features was based on the presence, absence or relative amounts of different glycans in the glycoprofile of all the training samples. The second group of features was based on a set of common peaks that were found across all the different glycoprofiles in the training samples. The intensity ratios of these common peaks were generated as features. The third group of features was generated by combining the set of common peaks based on glycan structural attributes such as branching and fucosylation.

Different types of classifiers have been developed and used in applications to generate patterns that are able to discriminate between two states.²⁴ For this study, the Rule Induction-based classifier was chosen for its advantage of generating “IF-THEN” rules, which allow the results of the classifiers to be explained in an easier manner compared to the other statistical or mathematical methods (e.g., genetic algorithms and neural networks). The Rule Induction classifier generates patterns in the form of, for example, “IF [(A>a) & (B<b) & (C=c)] or [(E>e) & (F<f)] THEN Disease State”, where A, B, C, D, E and F are extracted features and a, b, c, d, e and f are constants. Specifically, a modified version of the Rule Induction method described by Weiss, et.al.²⁵ was used to generate the rules (or patterns) to discriminate between populations.

### Classifying Human Prostate Cancer Through Glycomic Analysis

The PSA test is a widely used non-invasive measurement for prostate cancer. However, due to increased serum PSA levels in other inflammatory prostatic diseases, the test could suffer from high false-positive rates when using the established PSA cutoff of 4 ng/ml.²⁶ Although modifications to the test have been recently introduced (PSA density, PSA velocity, free PSA, complex to total PSA ratio, etc.),²⁷ the method still suffers from low predictive values when PSA levels are between 4 and 10 ng/mL.²⁶ Furthermore, increasing evidence of a high percentage of prostate cancer patients displaying PSA levels lower than 4 ng/mL is now starting to emerge.^{28,29}

The validity of the developed method to serve as a reliable tool for the discovery of signatures for prostate cancer was investigated. The sialylated N-glycoprofiles from the serum of prostate cancer (PCa) patients were compared to BPH and healthy donors. In order to minimize variations in the glycomic patterns resulting from other patient characteristics, samples were acquired from Genomics Collaborative, Inc. (Cambridge, Mass.) with matched controls to the PCa and BPH samples (Table 1).

TABLE 1
Demographics for the samples.

| | |
|---|---|
| Total | 142 |
| Pca | 33 |
| Pca White/Caucasian | 29 |
| Pca Black/African-American | 3 |
| Hispanic/Latino | 1 |
| BPH | 38 |
| White/Caucasian | 30 |
| BPH Black/African-American | 7 |
| Hispanic/Latino | 1 |
| Pca Control-Normal | 33 |
| Pca Control white | 29 |
| Pca Control Black | 3 |
| Pca Control Hispanic | 1 |
| BPH Control-Normal | 38 |
| BPH Control White | 30 |
| BPH Control Black | 7 |
| BPH Control Hispanic | 1 |

To use a population of statistical significance, the sialylated glycome of 166 serum samples were analyzed. Twenty-four of these samples were introduced as controls to monitor the variation of the method between samples and runs. The remaining 142 samples used to perform the glycomic pattern analysis were composed of 33 PCa samples, 38 BPH samples and their respective 71 matched controls. Two thirds of the samples (95) were used to build the rule-induction classifier. The remaining 47 samples were used to test the different rules that were generated.

On average, 60 peaks were detected across the different glycoprofiles. Three different categories of qualitative and quantitative features were extracted. The first type of extracted feature was the presence or absence of different glycans in a glycoprofile. For this qualitative feature, approximately 960 peaks were considered. The next two types of features were quantitative. The second type of feature comprised the normalized amplitudes of 22 peaks that were identified as common signals across all glycoprofiles (Table 2).

TABLE 2
Common signals across all glycoprofiles.

| Observed | Expected | |
|---|---|---|
| [M-H]⁻ | [M-H]⁻ | Composition |
| 1932 | 1933 | NeuAc₁Hex₅HexNAc₄ |
| 2061 | 2061 | NeuAc₂Hex₄HexNAc₄ |
| 2078 | 2078 | NeuAc₁Fuc₁Hex₅HexNAc₄ |
| 2177 | 2176 | NeuAc₁Hex₅HexNAc₆ |
| 2223 | 2223 | NeuAc₂Hex₅HexNAc₄ |
| 2323 | 2322 | NeuAc₁Fuc₁Hex₄HexNAc₆ |
| 2370 | 2369 | NeuAc₂Fuc₁Hex₅HexNAc₄ |
| 2426 | 2426 | NeuAc₂Hex₅HexNAc₅ |
| 2572 | 2572 | NeuAc₂Fuc₁Hex₅HexNAc₅ |
| 2588 | 2588 | NeuAc₂Hex₆HexNAc₅ |
| 2735 | 2735 | NeuAc₂Fuc₁Hex₆HexNAc₅ |
| 2834 | 2834 | NeuAc₁Fuc₂Hex₅HexNAc₇ |
| 2879 | 2880 | NeuAc₃Hex₆HexNAc₅ |
| 2953 | 2954 | NeuAc₂Hex₇HexNAc₆ |
| 2980 | 2980 | NeuAc₁Fuc₃Hex₅HexNAc₇ |
| 3026 | 3026 | NeuAc₃Fuc₁Hex₆HexNAc₅ |
| 3228 | 3229 | NeuAc₃Fuc₁Hex₆HexNAc₆ |
| 3245 | 3245 | NeuAc₃Hex₇HexNAc₆ |
| 3391 | 3393 | NeuAc₁Hex₉HexNAc₈ |
| 3536 | 3536 | NeuAc₄Hex₇HexNAc₆ |
| 3682 | 3682 | NeuAc₄Fuc₁Hex₇HexNAc₆ |
| 3902 | 3902 | NeuAc₄Hex₈HexNAc₇ |

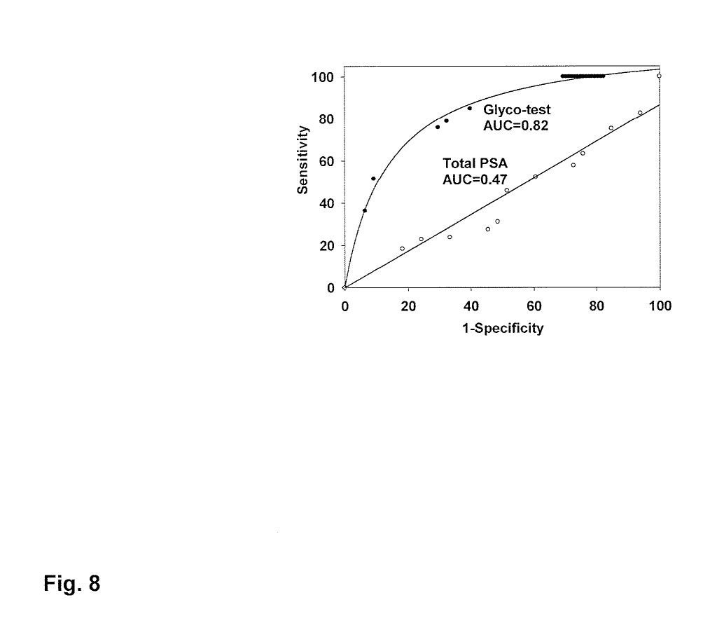

From the feature extraction process, 231 ratios of all combinations of the 22 peaks were extracted from each glycoprofile. The third type of feature generated combined the 22 common peaks into other features based on glycan attributes, such as the level of branching and fucosylation. For example, the common peaks corresponding to glycans with tetra-antennary structures were combined into one group and glycans with bi-antennary structures were combined into a different group. Ratios of these features based on glycan attributes, such as ratio of fucosylated to non-fucosylated structures, were also generated. Using these features, several rules were obtained from the Rule Induction-based classifier. One specific rule stood out when applied to the independent testing sample set: |D/A|≧8.9 and |C/B|≧2.1, where A corresponds to a glycan with molecular composition NeuAc₂Hex₅HexNAc₅ and 2426 [M-H]⁻, B is a glycan with NeuAc₂Hex₆HexNAc₅ molecular composition and 2588 [M-H]⁻, C is a NeuAc₃Fuc₁Hex₆HexNAc₅ glycan with 3026 [M-H]⁻ and D is a glycan with NeuAc₃Hex₇HexNAc₆ molecular composition and 3245 [M-H]⁻. This rule was able to segregate cancer from non-cancer patients with a sensitivity of 79% and a specificity of 68% (AUC=0.82) (FIG. 8). It was also observed that adding an additional parameter to this two-variable rule, resulted in a decrease in sensitivity to 76% but an increase in specificity to 71%, (AUC=0.82): |D/A|≧8.9 and |C/B|≧2.1 and |E/C|≧0.1, where E is a glycan NeuAc₁Hex₉HexNAc₈ molecular composition and 3391 [M-H]⁻. To compare the method to the standard surrogate used for prostate cancer diagnosis (PSA), the total PSA serum levels of the samples used in this study were measured. The developed method showed better predictive values in comparison to the total PSA levels measured using standard ELISA tests. Using the established 4 ng/mL PSA cutoff value for prostate cancer, this test showed a sensitivity of 49% and a specificity of 69% (AUC=0.47) (FIG. 8). The low sensitivity displayed by this test correlates with the increasing evidence of a high percentage of prostate cancer patients displaying PSA levels lower than 4 ng/mL or usual complications of protein precipitation that can affect this test.^{28,29}

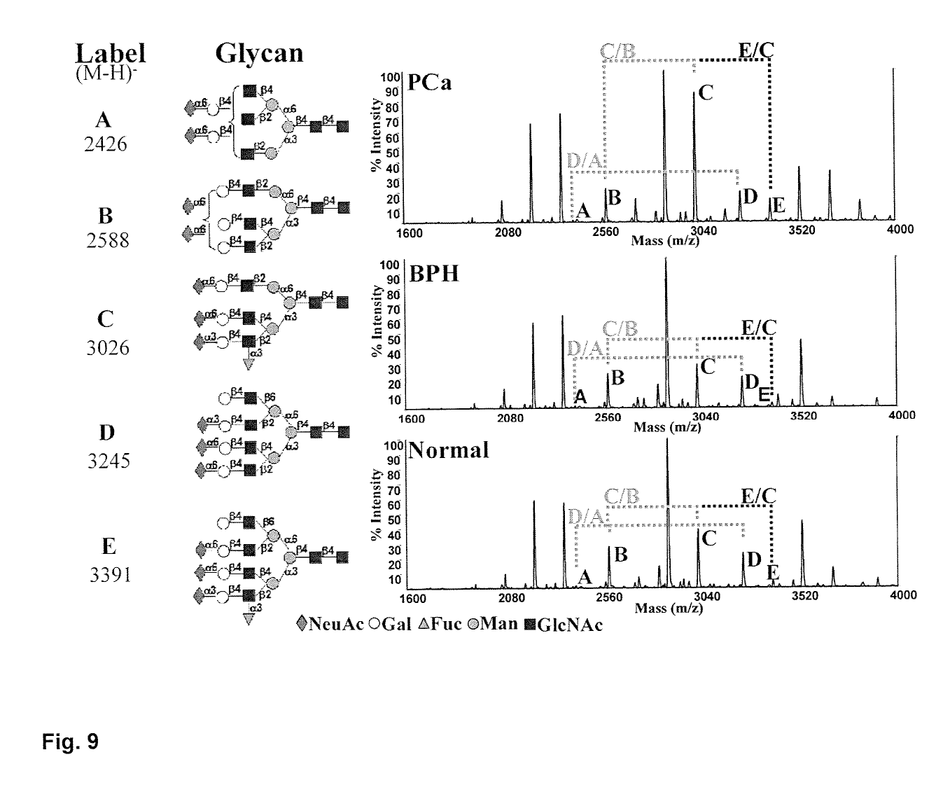

Visually inspecting the MALDI-TOF-MS spectra of the serum glycoprofiles from different patients, the recognized patterns determined by the bioinformatics platform were observed. FIG. 9 illustrates the comparison between representative MS spectra from PCa, BPH and control patients and shows the glycomic patterns obtained from the bioinformatics analysis that segregate prostate cancer patients from BPH and controls. Two particularly interesting observation from these identified patterns is the increased expression of the sialylated structures C and E containing the sialyl Lewis X epitope and the increased branching structures D and E in samples from prostate cancer patients. Evidence has shown the association of the sialyl Lewis X epitope with the intricate stages of tumor progression. For example, the overexpression of sialyl Lewis X has been shown to facilitate the extravasation of cancer cells during hematogenous metastasis via their interaction with selectin receptors.³⁰⁻³² Additionally, the HPLC profile of serum glycans from a cancer patient has been compared to a pooled serum sample, and the same overexpression of structures C was observed.¹⁸ The results illustrate the advantage of monitoring global glycomic patterns and validate this method as a reliable tool for the identification of cancer glycomic patterns.

Increased branching has been correlated with tumor invasion, angiogenesis and metastasis.^{12, 15, 32} Also, increased branching on PSA has been described as a glycosylation alteration associated with prostate cancer.³⁵ Based on the features that captured ratios of glycans with different levels of branching, it was observed that more PCa cancer patients displayed high relative ratios of tetra-antennary to bi-antennary structures when compared to BPH and control patients. The average ratio of tetra-antennary to bi-antennary structures for the control population was 0.6. When a threshold of 0.8 (33% above the average value for the controls) was considered, 49% of the PCa samples showed elevated relative ratios of tetra-antennary to bi-antennary structures while 22% of BPH patients and 10% of normal patients showed high tetra-antennary to bi-antennary ratios. These results show that increased branching of serum N-linked glycans is correlated with prostate cancer.

Whether the identified signatures could have any correlation with the progression of cancer was also tested. Out of 33 PCa samples used to test the different rules generated from the bioinformatics platform, 1 sample was Stage I, 26 were Stage II and 6 samples were Stage III. It was observed that 83% (5 out of 6) of Stage III patients had D/A>9.8 and C/B>3.5. On the other hand, only 11% (3 out of 27) of the Stage I and II (combined) obeyed this rule. Also, 83% (5 out of 6) of Stage III have tetra-antennary/bi-antennary ratios >0.8 while 41% (11 out of 27) of the Stage I and II (combined) have tetra-antennary/bi-antennary ratios >0.8. These results suggest that the glycan ratios C/B and D/A as well as the tetra-antennary/bi-antennary ratios have a correlation with cancer progression. These ratios are both higher in cancer when compared to non-cancer patients and they seem to be higher for Stage III PCa patients when compared to patients with earlier stages of cancer.

### Characterization of Glycans in the Glycomic Pattern

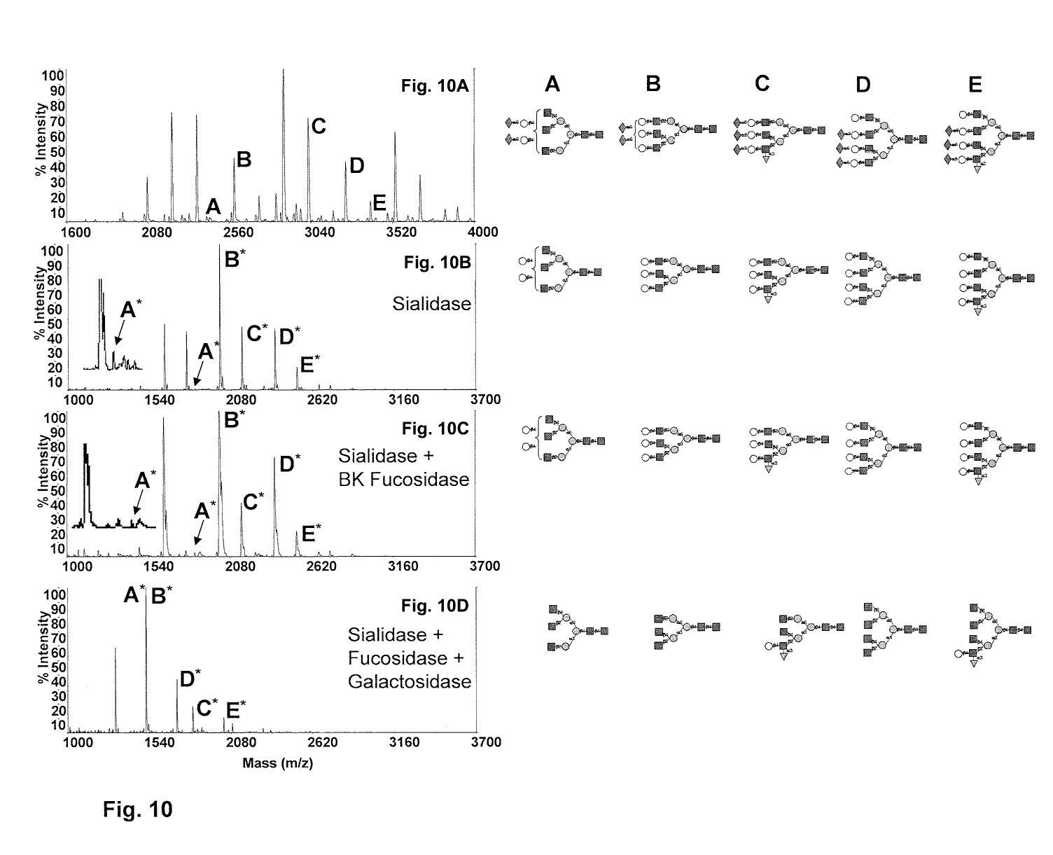

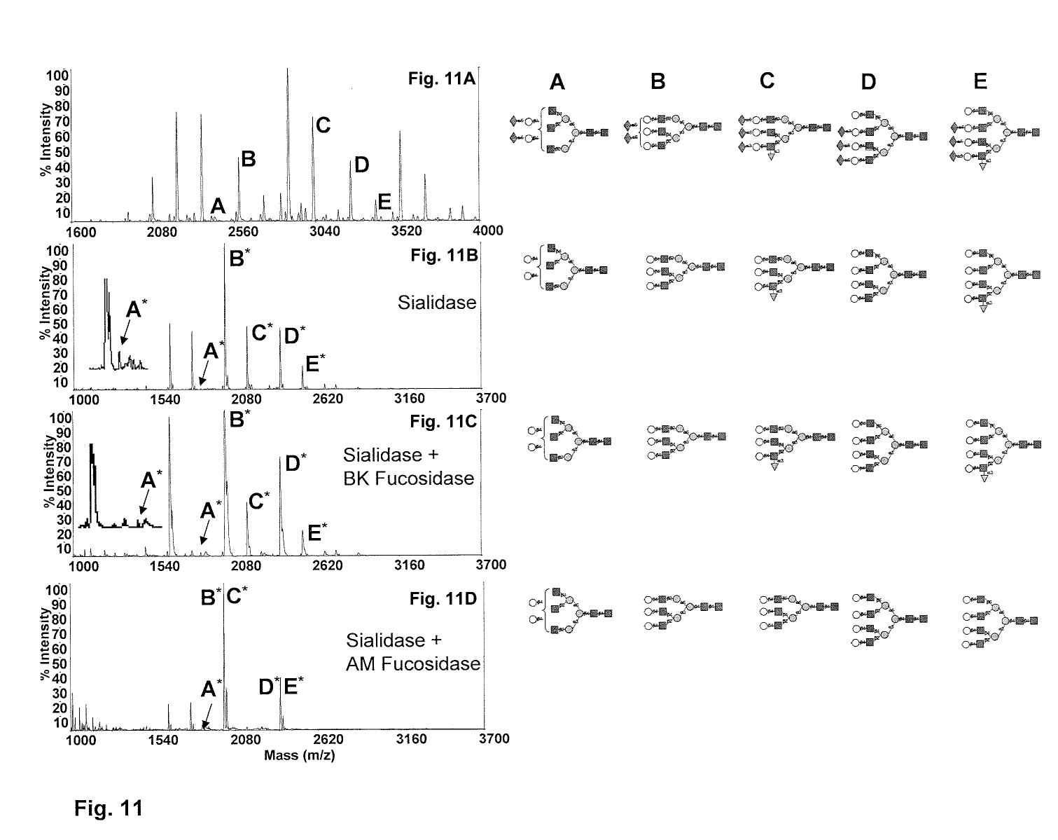

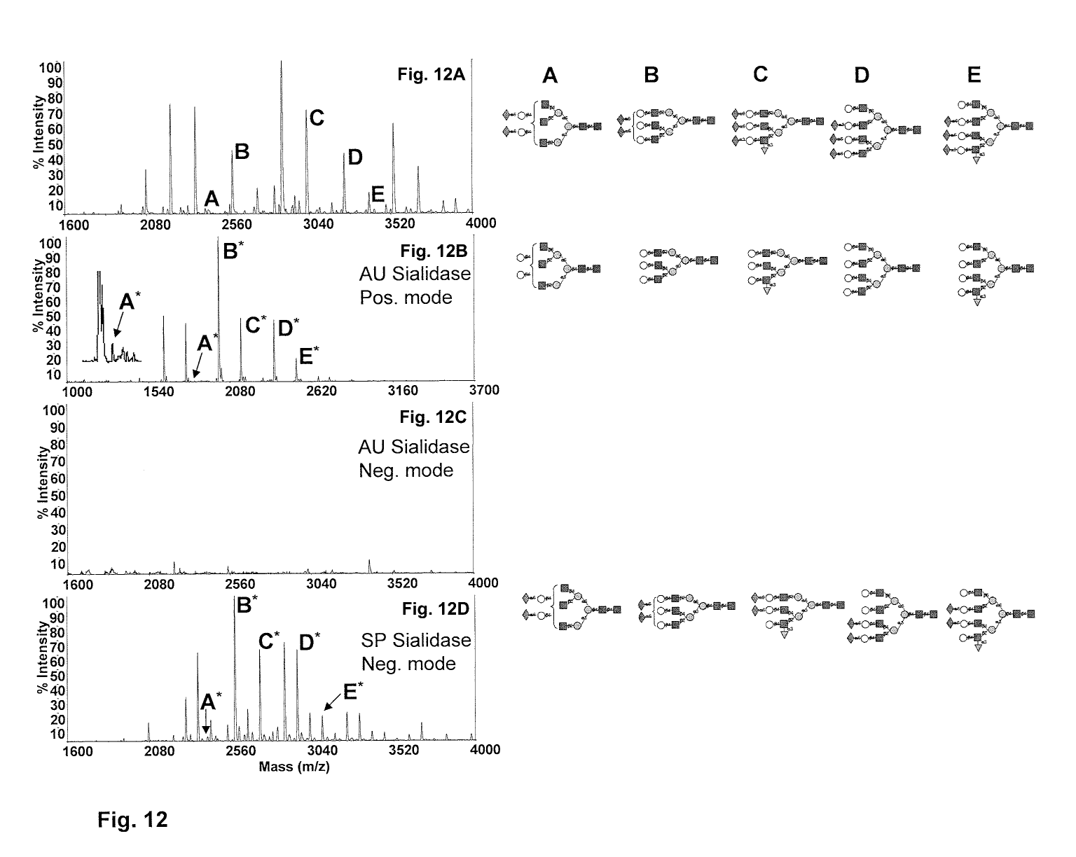

A panel of glycosidases was used to further characterize the glycans involved in the glycomic pattern identified by the bioinformatic analysis. Orthogonal fucosidases with different substrate specificity were used to confirm the linkage and position of fucoses within the glycan. For example, bovine kidney fucosidase releases α-1,6 core-linked fucoses more efficiently than other fucoses. On the other hand, almond meal fucosidase is specific for α-1,3,4-linked fucoses. As shown in FIG. 10 and FIG. 11, glycans C and E are resistant to cleavage with bovine kidney fucosidase and sensitive to almond meal fucosidase. These structures were further confirmed by the additional treatment of the glycans with jack bean β-galactosidase, which was unable to cleave terminal galactoses linked to GlcNac residues containing an α-1,3-linked fucose (FIG. 10d). The sialic acid linkage was also determined using a combination of non-specific Arthobacter ureafaciens sialidase and Streptococcus pneumoniae sialidase, which is specific for α-2,3-linked sialic acids (FIG. 12). Some of these glycans have been characterized using a different method.^{1,2}

### Conclusion

Whether alterations to global glycomic patterns expressed in the serum of patients could reflect some of the complex glycan remodeling associated with cancer has been assessed. Patterns could capture some of the alterations to the complex network of glycosyltransferases and thus serve as sensitive biomarkers for cancer diagnostic purposes. To capture these glycomic alterations, a method that efficiently identifies glycan patterns associated with cancer was used. The rapid analysis provided by the combination of MALDI-TOF-MS and a glycan-focused bioinformatics platform allowed for the analysis of a statistically significant sample population. By focusing on the alterations to global glycomic patterns, instead of individual glycoproteins, this approach overcomes some of the challenges arising from the pleiotropic effects of glycan remodeling.

When studying the overall glycan profiles from serum, the analyzed glycans could arise from a mixture of high and low abundance proteins. As most abundant glycoproteins (such as IgGs and transferrin) are usually not highly sialylated, however, it is expected that the cancer-associated glycan patterns would not reflect alterations of these high abundance glycoproteins. Although some of the glycans identified from the overall profile (Table 2), could be from high abundance glycoproteins, it was interesting that the cancer-associated glycans identified by the informatics platform do not correlate with glycans from IgGs or transferrin. Also, removal of IgGs from the serum prior to analysis mainly affected the signals of neutral glycans species but did not significantly affect signals of the acidic species. It is also interesting that one of the primary glycans found in the PCa glycomic pattern (glycan C) has been shown to be overexpressed in PSA from prostate cancer patients.¹⁴ However, it is difficult to say whether some of these glycan signatures are in fact a reflection of PSA's glycan alteration or from other glycoproteins (or a mixture of glycoproteins).

While no individual glycan alone gave a reliable diagnostic signature, segregation of the cancer from the non-cancer population was observed for patterns involving more than one glycan. This may reflect the fact that the alteration to glycan biosynthesis occurs through an interconnected circuit that can affect not only one, but multiple glycosyltransferases. These results further illustrate the importance of global glycomic patterns as diagnostic fingerprints. The fact that increased branching was observed as one of the main trends in the PCa patient group correlates with the increased activity of N-acetylglucosaminyltransferase-V in cancer. Furthermore, the increased expression of sialyl Lewis X epitopes in PCa patients could be associated with the increased activity of α1,3-fucosyltransferase in cancer.

### Example 2

### Diagnostic Glycomic Patterns for Multiple Myeloma

To determine whether the method could identify glycan signatures associated with multiple myeloma, the acidic glycoprofiles of 71 multiple myeloma patients were analyzed in comparison to the 71 healthy patients. On average, 60 peaks were detected across the different glycoprofiles. Two different categories of qualitative and quantitative features were extracted.

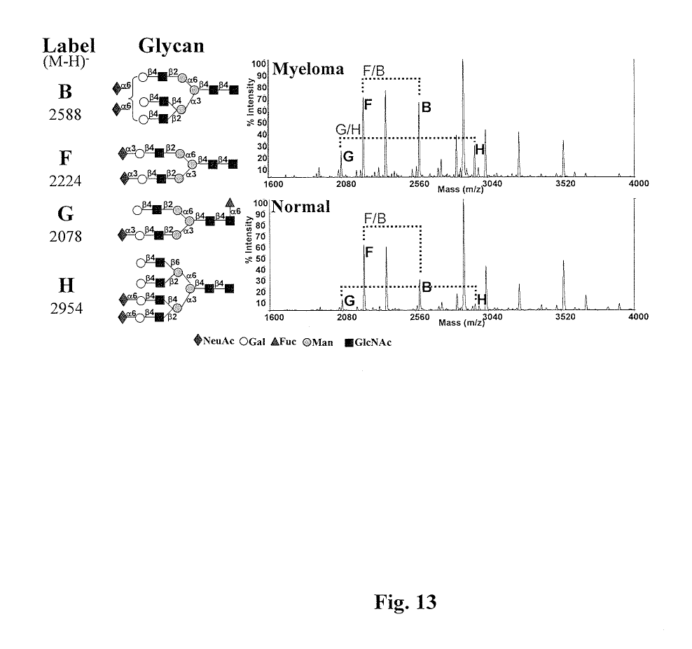

The first type of extracted feature was the presence or absence of different glycans in a glycoprofile. The next type of feature was quantitative. This feature comprised the normalized amplitudes of 22 peaks that were identified as common signals across all glycoprofiles (Table 3). From the feature extraction process, 231 ratios of combinations of the 22 peaks were extracted from each glycoprofile. Using these features, several rules were obtained from the rule induction-based classifier. An example of a specific rule that stood out when applied to the independent testing sample set is |F/B|≦2.3 and |G/H|≦2.3, where B corresponds to a glycan with molecular composition NeuAc₂Hex₆HexNAc₅ with 2588 [M-H]⁻, F is a NeuAc₂Hex₅HexNAc₄ glycan with 2224 [M-H]⁻, G is a glycan with molecular composition NeuAc₁Fuc₁Hex₅HexNAc₄ with 2078 [M-H]⁻, and H is a glycan with NeuAc₂Hex₇HexNAc₆ molecular composition with 2954 [M-H]⁻. This rule was able to segregate multiple myeloma patients from non-cancer patients with sensitivity of 79% and specificity of 70% (AUC=0.82). These signatures can be easily observed by visually inspecting the MS glycoprofiles of multiple myeloma patients in comparison to non-cancer patients (FIG. 13).

Interestingly, the obtained patterns that segregated the multiple myeloma patients from the non-cancer patients were different from those obtained for the previous prostate cancer patients. However, they also showed a similar increasing branching trend observed for the PCa patients (a signature associated with invasion, angiogenesis and metastasis).

TABLE 3
Molecular composition assignment of N-glycans based
on [M-H]⁻ ions of the 22 common species in the
MALDI-TOF-MS glycoprofiles.

| Observed | Expected | |
|---|---|---|
| [M-H]⁻ | [M-H]⁻ | Composition |
| 1932 | 1933 | NeuAc₁Hex₅HexNAc₄ |
| 2061 | 2061 | NeuAc₂Hex₄HexNAc₄ |
| 2078 | 2078 | NeuAc₁Fuc₁Hex₅HexNAc₄ |
| 2177 | 2176 | NeuAc₁Hex₅HexNAc₆ |
| 2223 | 2223 | NeuAc₂Hex₅HexNAc₄ |
| 2323 | 2322 | NeuAc₁Fuc₁Hex₄HexNAc₆ |
| 2370 | 2369 | NeuAc₂Fuc₁Hex₅HexNAc₄ |
| 2426 | 2426 | NeuAc₂Hex₅HexNAc₅ |
| 2572 | 2572 | NeuAc₂Fuc₁Hex₅HexNAc₅ |
| 2588 | 2588 | NeuAc₂Hex₆HexNAc₅ |
| 2735 | 2735 | NeuAc₂Fuc₁Hex₆HexNAc₅ |
| 2834 | 2834 | NeuAc₁Fuc₂Hex₅HexNAc₇ |
| 2879 | 2880 | NeuAc₃Hex₆HexNAc₅ |
| 2953 | 2954 | NeuAc₂Hex₇HexNAc₆ |
| 2980 | 2980 | NeuAc₁Fuc₃Hex₅HexNAc₇ |
| 3026 | 3026 | NeuAc₃Fuc₁Hex₆HexNAc₅ |
| 3228 | 3229 | NeuAc₃Fuc₁Hex₆HexNAc₆ |
| 3245 | 3245 | NeuAc₃Hex₇HexNAc₆ |
| 3391 | 3393 | NeuAc₁Hex₉HexNAc₈ |
| 3536 | 3536 | NeuAc₄Hex₇HexNAc₆ |
| 3682 | 3682 | NeuAc₄Fuc₁Hex₇HexNAc₆ |
| 3902 | 3902 | NeuAc₄Hex₈HexNAc₇ |

### REFERENCES

The listing of the references in the following list is not intended to be an admission that any of the references is a prior art reference.
1. Garbis, S., Lubec, G. & Fountoulakis, M. Limitations of current proteomics technologies. J Chromatogr A 1077, 1-18 (2005).
2. Dube, D. H. & Bertozzi, C. R. Glycans in cancer and inflammation—potential for therapeutics and diagnostics. Nat Rev Drug Discov 4, 477-488 (2005).
3. Fuster, M. M. & Esko, J. D. The sweet and sour of cancer: glycans as novel therapeutic targets. Nat Rev Cancer 5, 526-542 (2005).
4. Yang, Z. & Hancock, W. S. Approach to the comprehensive analysis of glycoproteins isolated from human serum using a multi-lectin affinity column. J Chromatogr A 1053, 79-88 (2004).
5. Carlson, J., Eriksson, S., Alm, R. & Kjellstrom, T. Biosynthesis of abnormally glycosylated alpha 1-antitrypsin by a human hepatoma cell line. Hepatology 4, 235-241 (1984).
6. Hanley, J. M., Haugen, T. H. & Heath, E. C. Biosynthesis and processing of rat haptoglobin. J Biol Chem 258, 7858-7869 (1983).
7. Hahn, T. J. & Goochee, C. F. Growth-associated glycosylation of transferrin secreted by HepG2 cells. J Biol Chem 267, 23982-23987 (1992).
8. Ghosh, S., Hevi, S. & Chuck, S. L. Regulated secretion of glycosylated human ferritin from hepatocytes. Blood 103, 2369-2376 (2004).
9. Polascik, T. J., Oesterling, J. E. & Partin, A. W. Prostate specific antigen: a decade of discovery-what we have learned and where we are going. J Urol 162, 293-306 (1999).
10. Jacobs, I. J. & Menon, U. Progress and challenges in screening for early detection of ovarian cancer. Mol Cell Proteomics 3, 355-366 (2004).
11. Girnita, L. et al. Inhibition of N-linked glycosylation down-regulates insulin-like growth factor-1 receptor at the cell surface and kills Ewing's sarcoma cells: therapeutic implications. Anticancer Drug Des 15, 67-72 (2000).
12. Dennis, J. W., Laferte, S., Waghorne, C., Breitman, M. L. & Kerbel, R. S. Beta 1-6 branching of Asn-linked oligosaccharides is directly associated with metastasis. Science 236, 582-585 (1987).
13. Kim, Y. J. & Varki, A. Perspectives on the significance of altered glycosylation of glycoproteins in cancer. Glycoconj J 14, 569-576 (1997).
14. Seidenfaden, R., Krauter, A., Schertzinger, F., Gerardy-Schahn, R. & Hildebrandt, H. Polysialic acid directs tumor cell growth by controlling heterophilic neural cell adhesion molecule interactions. Mol Cell Biol 23, 5908-5918 (2003).
15. Guo, H. B., Lee, I., Kamar, M., Akiyama, S. K. & Pierce, M. Aberrant N-glycosylation of beta1 integrin causes reduced alpha5beta1 integrin clustering and stimulates cell migration. Cancer Res 62, 6837-6845 (2002).
16. Kui Wong, N. et al. Characterization of the oligosaccharides associated with the human ovarian tumor marker CA125. J Biol Chem 278, 28619-28634 (2003).
17. Peracaula, R. et al. Altered glycosylation pattern allows the distinction between prostate-specific antigen (PSA) from normal and tumor origins. Glycobiology 13, 457-470 (2003).
18. Tabares, G. et al. Different glycan structures in prostate-specific antigen (PSA) from prostate cancer sera in relation to seminal plasma PSA. Glycobiology published online (2005).
19. Petricoin, E. F. et al. Use of proteomic patterns in serum to identify ovarian cancer. Lancet 359, 572-577 (2002).
20. Wulfkuhle, J. D., Liotta, L. A. & Petricoin, E. F. Proteomic applications for the early detection of cancer. Nat Rev Cancer 3, 267-275 (2003).
21. Conrads, T. P., Zhou, M., Petricoin, E. F., 3rd, Liotta, L. & Veenstra, T. D. Cancer diagnosis using proteomic patterns. Expert Rev Mol Diagn 3, 411-420 (2003).
22. Adam, B. L. et al. Serum protein fingerprinting coupled with a pattern-matching algorithm distinguishes prostate cancer from benign prostate hyperplasia and healthy men. Cancer Res 62, 3609-3614 (2002).
23. Qu, Y. et al. Boosted decision tree analysis of surface-enhanced laser desorption/ionization mass spectral serum profiles discriminates prostate cancer from noncancer patients. Clin Chem 48, 1835-1843 (2002).
24. Weiss, S. & Indrukhya, N. Predictive data mining-A practical guide. (San Francisco; 1998).
25. Weiss, S. & Indurkhya, N. Optimized rule induction. IEEE Expert Journal 8, 61-69 (1993).
26. Brawer, M. K. Prostate-specific antigen: current status. CA Cancer J Clin 49, 264-281 (1999).
27. Djavan, B. et al. PSA, PSA density, PSA density of transition zone, free/total PSA ratio, and PSA velocity for early detection of prostate cancer in men with serum PSA 2.5 to 4.0 ng/mL. Urology 54, 517-522 (1999).
28. Djavan, B., Fong, Y. K., Remzi, M., Fakhori, M. & Marberger, M. New Serum and Urinary Markers for Prostate Cancer Detection in the New Millennium. Eur. Urol. Suppl. 3, 25-32 (2004).
29. Thompson, I. M. et al. Prevalence of prostate cancer among men with a prostate-specific antigen level < or =4.0 ng per milliliter. N Engl J Med 350, 2239-2246 (2004).
30. Kannagi, R. Carbohydrate-mediated cell adhesion involved in hematogenous metastasis of cancer. Glycoconj J 14, 577-584 (1997).
31. Kannagi, R. Molecular mechanism for cancer-associated induction of sialyl Lewis X and sialyl Lewis A expression—The Warburg effect revisited. Glycoconj J 20, 353-364 (2004).
32. Jorgensen, T. et al. Up-regulation of the oligosaccharide sialyl LewisX: a new prognostic parameter in metastatic prostate cancer. Cancer Res 55, 1817-1819 (1995).
33. Seelentag, W. K. et al. Expression of CD44 isoforms and beta 1,6-branched oligosaccharides in human malignant melanoma is correlated with tumor progression but not with metastatic potential. J Cutan Pathol 24, 206-211 (1997).
34. Papac, D. I., Briggs, J. B., Chin, E. T. & Jones, A. J. A high-throughput microscale method to release N-linked oligosaccharides from glycoproteins for matrix-assisted laser desorption/ionization time-of-flight mass spectrometric analysis. Glycobiology 8, 445-454 (1998).
35. Parakash, S. & Robbins, P. W. Glycotyping of prostate specific antigen. Glycobiology 10, 173-176 (2000).

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

I/We claim:

1. A method for diagnosing, comprising:
obtaining a sample from a subject,
determining the amount of a first glycan selected from the group consisting of a NeuAc1 Hex5HexNAc4 glycan, a NeuAc2Hex4HexNAc4 glycan, a NeuAc1Fuc1Hex5HexNAc4 glycan, a NeuAc1Hex5HexNAc6, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
determining the amount of a second glycan selected from the group consisting of NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1 Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
calculating the relative ratio of the first glycan and the second glycan, and
comparing the relative ratio of the first glycan and the second glycan to a first threshold value.

2. The method of claim 1, wherein the method further comprises:
determining the amount of a third glycan selected from the group consisting of a NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
determining the amount of a fourth glycan selected from the group consisting of NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
calculating the relative ratio of the third glycan and the fourth glycan, and
comparing the relative ratio of the third glycan and the fourth glycan to a second threshold value.

3-7. (canceled)

8. The method of claim 2, wherein the method further comprises:
determining the amount of a fifth glycan selected from the group consisting of a NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan⁻, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
determining the amount of a sixth glycan selected from the group consisting of NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
calculating the relative ratio of the fifth glycan and the sixth glycan, and
comparing the relative ratio of the fifth glycan and the sixth glycan to a third threshold value.

9-13. (canceled)

14. The method of claim 2, wherein the first glycan is a NeuAc2Hex5HexNAc5 glycan, the second glycan is NeuAc3Hex7HexNAc6 glycan, the third glycan is a NeuAc3Hex6HexNAc5 glycan, and the fourth glycan is a NeuAc4Hex7HexNAc6 glycan.

15. The method of claim 14, wherein the method further comprises:
determining the amount of a fifth glycan selected from the group consisting of a NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
determining the amount of a sixth glycan selected from the group consisting of NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
calculating the relative ratio of the fifth glycan and the sixth glycan, and
comparing the relative ratio of the fifth glycan and the sixth glycan to a third threshold value.

16-17. (canceled)

18. The method of claim 1, wherein the method further comprises arriving at a final diagnosis.

19. The method of claim 1, wherein the method further comprises performing an additional diagnostic test on the subject.

20-22. (canceled)

23. The method of claim 1, wherein the subject is suspected of having cancer.

24-25. (canceled)

26. The method of claim 19, wherein the additional diagnostic test comprises:
determining the amounts of one or more glycans in the sample, and
comparing the amounts with a threshold value.

27. The method of claim 19, wherein the additional diagnostic test comprises:
determining the amounts of two or more glycans in the sample,
calculating at least one relative ratio of the two or more glycans, and
comparing the at least one relative ratio with a threshold value.

28-29. (canceled)

30. The method of claim 19, wherein the additional diagnostic test, comprises:
determining the relative ratio of tetra-antennary glycans to bi-antennary glycans, and
comparing the relative ratio to a threshold value.

31-33. (canceled)

34. The method of claim 19, wherein the additional diagnostic test, comprises:
determining the amount of a prostate cancer-specific marker in the sample, and
comparing the amount of the prostate cancer-specific marker to a threshold value.

35. (canceled)

36. The method of claim 19, wherein the additional diagnostic test, comprises:
determining the amount of a multiple myeloma-specific marker in the sample, and
comparing the amount of the multiple myeloma-specific marker to a threshold value.

37. (canceled)

38. A method for diagnosing, comprising:
obtaining a sample from a subject,
determining the relative ratio of tetra-antennary glycans to bi-antennary glycans in the sample,
and comparing the relative ratio to a threshold value.

39-49. (canceled)

50. A method for analyzing one or more samples, comprising:
determining the amount of two or more glycans selected from the group consisting of a NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the one or more samples, and
calculating relative ratios of the glycan amounts in the samples.

51. The method of claim 50, further comprising determining one or more threshold values from the relative ratios.

52-73. (canceled)

74. A method for analyzing one or more samples, comprising:
determining the amount of tetra-antennary glycans and bi-antennary glycans in the samples,
calculating relative ratios of tetra-antennary glycans to bi-antennary glycans in the samples, and
determining one or more threshold values from the relative ratios.

75-83. (canceled)

84. A method for determining the stage of cancer, comprising:
obtaining a sample from a subject,
determining the amount of a first glycan selected from the group consisting of a NeuAc1 Hex5HexNAc4 glycan, a NeuAc2Hex4HexNAc4 glycan, a NeuAc1Fuc1Hex5HexNAc4 glycan, a NeuAc1Hex5HexNAc6, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
determining the amount of a second glycan selected from the group consisting of NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
calculating the relative ratio of the first glycan and the second glycan, and
comparing the relative ratio of the first glycan and the second glycan to a first threshold value.

85. The method of claim 84, wherein the method further comprises:
determining the amount of a third glycan selected from the group consisting of a NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
determining the amount of a fourth glycan selected from the group consisting of NeuAc1 Hex5HexNAc4 glycan, a NeuAc2Hex4HexNAc4 glycan, a NeuAc1Fuc1Hex5HexNAc4 glycan, a NeuAc1Hex5HexNAc6 glycan, a NeuAc2Hex5HexNAc4 glycan, a NeuAc1Fuc1Hex4HexNAc6 glycan, a NeuAc2Fuc1Hex5HexNAc4 glycan, a NeuAc2Hex5HexNAc5 glycan, a NeuAc2Fuc1Hex5HexNAc5 glycan, a NeuAc2Hex6HexNAc5 glycan, a NeuAc2Fuc1Hex6HexNAc5 glycan, a NeuAc1Fuc2Hex5HexNAc7 glycan, a NeuAc3Hex6HexNAc5 glycan, a NeuAc2Hex7HexNAc6 glycan, a NeuAc1Fuc3Hex5HexNAc7 glycan, a NeuAc3Fuc1Hex6HexNAc5 glycan, a NeuAc3Fuc1Hex6HexNAc6 glycan, a NeuAc3Hex7HexNAc6 glycan, a NeuAc1Hex9HexNAc8 glycan, a NeuAc4Hex7HexNAc6 glycan, a NeuAc4Fuc1Hex7HexNAc6 glycan and a NeuAc4Hex8HexNAc7 glycan in the sample,
calculating the relative ratio of the third glycan and the fourth glycan, and
comparing the relative ratio of the third glycan and the fourth glycan to a second threshold value.

86-88. (canceled)

89. A method for determining the stage of cancer, comprising:
obtaining a sample from a subject,
determining the relative ratio of tetra-antennary glycans to bi-antennary glycans in the sample,
and comparing the relative ratio to a threshold value to determine the stage of cancer in the subject.

90-91. (canceled)
